SEQUENCE LISTING (60 sequences)

SEQ ID NO: 1 - RNA, 21 nt, Artificial Sequence
  Description of Artificial Sequence Synthetic oligonucleotide
ucccugagaccucaaguguga

SEQ ID NO: 2 - RNA, 22 nt, Artificial Sequence
  Description of Artificial Sequence Synthetic oligonucleotide
ucaccggguguaaaucagcuug

SEQ ID NO: 3 - RNA, 22 nt, Artificial Sequence
  Description of Artificial Sequence Synthetic oligonucleotide
uagcagcacguaaauauuggcg

SEQ ID NO: 4 - RNA, 22 nt, Artificial Sequence
  Description of Artificial Sequence Synthetic oligonucleotide
ucccugagacccuaacuuguga

SEQ ID NO: 5 - RNA, 22 nt, Artificial Sequence
  Description of Artificial Sequence Synthetic oligonucleotide
ucagugcacuacagaacuuugu

SEQ ID NO: 6 - RNA, 22 nt, Artificial Sequence
  Description of Artificial Sequence Synthetic oligonucleotide
agagguaguagguugcauaguu

SEQ ID NO: 7 - RNA, 22 nt, Artificial Sequence
  Description of Artificial Sequence Synthetic oligonucleotide
ugagguaguaguuuguacaguu

SEQ ID NO: 8 - RNA, 22 nt, Artificial Sequence
  Description of Artificial Sequence Synthetic oligonucleotide
uagcagcacaucaugguuuaca

SEQ ID NO: 9 - RNA, 23 nt, Artificial Sequence
  Description of Artificial Sequence Synthetic oligonucleotide
aaaagugcuuacagugcagguag

SEQ ID NO: 10 - RNA, 23 nt, Artificial Sequence
  Description of Artificial Sequence Synthetic oligonucleotide
uguaguguuuccuacuuuaugga

SEQ ID NO: 11 - RNA, 23 nt, Artificial Sequence
  Description of Artificial Sequence Synthetic oligonucleotide
caacggaaucccaaaagcagcug

SEQ ID NO: 12 - RNA, 23 nt, Artificial Sequence
  Description of Artificial Sequence Synthetic oligonucleotide
cagugcaauaguauugucaaagc

SEQ ID NO: 13 - DNA, 10 bp, Artificial Sequence
  Description of Artificial Sequence Synthetic primer
gtctcaggga

SEQ ID NO: 14 - DNA, 19 bp, Artificial Sequence
  Description of Artificial Sequence Synthetic primer
ctcaagtgtgatccctgag

SEQ ID NO: 15 - DNA, 19 bp, Artificial Sequence
  Description of Artificial Sequence Synthetic primer
agggatcacacttgaggtc

SEQ ID NO: 16 - DNA, 10 bp, Artificial Sequence
  Description of Artificial Sequence Synthetic primer
atttacaccc

SEQ ID NO: 17 - DNA, 19 bp, Artificial Sequence
  Description of Artificial Sequence Synthetic primer
taaatcagcttgtcaccgg

SEQ ID NO: 18 - DNA, 19 bp, Artificial Sequence
  Description of Artificial Sequence Synthetic primer
gtgacaagctgatttacac

SEQ ID NO: 19 - DNA, 10 bp, Artificial Sequence
  Description of Artificial Sequence Synthetic primer
ctgctacgcc

SEQ ID NO: 20 - DNA, 18 bp, Artificial Sequence
  Description of Artificial Sequence Synthetic primer
agcacgtaaatattggcg

SEQ ID NO: 21 - DNA, 18 bp, Artificial Sequence
  Description of Artificial Sequence Synthetic primer
ccaatatttacgtgctgc

SEQ ID NO: 22 - DNA, 10 bp, Artificial Sequence
  Description of Artificial Sequence Synthetic primer
caagttaggg

SEQ ID NO: 23 - DNA, 17 bp, Artificial Sequence
  Description of Artificial Sequence Synthetic primer
gaccctaacttgtgatc

SEQ ID NO: 24 - DNA, 17 bp, Artificial Sequence
  Description of Artificial Sequence Synthetic primer
cacaagttagggtctca

SEQ ID NO: 25 - DNA, 10 bp, Artificial Sequence
  Description of Artificial Sequence Synthetic primer
gcactgaaca

SEQ ID NO: 26 - DNA, 20 bp, Artificial Sequence
  Description of Artificial Sequence Synthetic primer
tacagaactttgttcagtgc

SEQ ID NO: 27 - DNA, 20 bp, Artificial Sequence
  Description of Artificial Sequence Synthetic primer
ctgaacaaagttctgtagtg

SEQ ID NO: 28 - DNA, 10 bp, Artificial Sequence
  Description of Artificial Sequence Synthetic primer
ctactacctc

SEQ ID NO: 29 - DNA, 19 bp, Artificial Sequence
  Description of Artificial Sequence Synthetic primer
gttagaggtagtaggttgc

SEQ ID NO: 30 - DNA, 19 bp, Artificial Sequence
  Description of Artificial Sequence Synthetic primer
acctactacctctaactat

SEQ ID NO: 31 - DNA, 10 bp, Artificial Sequence
  Description of Artificial Sequence Synthetic primer
ctactacctc

SEQ ID NO: 32 - DNA, 21 bp, Artificial Sequence
  Description of Artificial Sequence Synthetic primer
gaggtagtagtttgtacagtt

SEQ ID NO: 33 - DNA, 21 bp, Artificial Sequence
  Description of Artificial Sequence Synthetic primer
tgtacaaactactacctcaaa

SEQ ID NO: 34 - DNA, 10 bp, Artificial Sequence
  Description of Artificial Sequence Synthetic primer
cctgcactgt

SEQ ID NO: 35 - DNA, 19 bp, Artificial Sequence
  Description of Artificial Sequence Synthetic primer
gcagcacatcatggtttac

SEQ ID NO: 36 - DNA, 20 bp, Artificial Sequence
  Description of Artificial Sequence Synthetic primer
aaccatgatgtgctgctatg

SEQ ID NO: 37 - DNA, 10 bp, Artificial Sequence
  Description of Artificial Sequence Synthetic primer
cctgcactgt

SEQ ID NO: 38 - DNA, 19 bp, Artificial Sequence
  Description of Artificial Sequence Synthetic primer
agaaaagtgcttacagtgc

SEQ ID NO: 39 - DNA, 19 bp, Artificial Sequence
  Description of Artificial Sequence Synthetic primer
ctgtaagcacttttctacc

SEQ ID NO: 40 - DNA, 10 bp, Artificial Sequence
  Description of Artificial Sequence Synthetic primer
ctacatccat

SEQ ID NO: 41 - DNA, 20 bp, Artificial Sequence
  Description of Artificial Sequence Synthetic primer
gtagtgtttcctactttatg

SEQ ID NO: 42 - DNA, 20 bp, Artificial Sequence
  Description of Artificial Sequence Synthetic primer
aaagtaggaaacactacatc

SEQ ID NO: 43 - DNA, 10 bp, Artificial Sequence
  Description of Artificial Sequence Synthetic primer
gggattccgt

SEQ ID NO: 44 - DNA, 18 bp, Artificial Sequence
  Description of Artificial Sequence Synthetic primer
caacggaatcccaaaagc

SEQ ID NO: 45 - DNA, 18 bp, Artificial Sequence
  Description of Artificial Sequence Synthetic primer
tttgggattccgttgcag

SEQ ID NO: 46 - DNA, 10 bp, Artificial Sequence
  Description of Artificial Sequence Synthetic primer
ctggctttga

SEQ ID NO: 47 - DNA, 20 bp, Artificial Sequence
  Description of Artificial Sequence Synthetic primer
tgcaatagtattgtcaaagc

SEQ ID NO: 48 - DNA, 20 bp, Artificial Sequence
  Description of Artificial Sequence Synthetic primer
ttgacaatactattgcactg

SEQ ID NO: 49 - DNA, 22 bp, Artificial Sequence
  Description of Artificial Sequence Synthetic oligonucleotide
cacttgaggtctcagggtatcg

SEQ ID NO: 50 - DNA, 22 bp, Artificial Sequence
  Description of Artificial Sequence Synthetic oligonucleotide
gctgatttacacccgggtatcg

SEQ ID NO: 51 - DNA, 25 bp, Artificial Sequence
  Description of Artificial Sequence Synthetic oligonucleotide
gccaatatttacgtgctgcgtatcg

SEQ ID NO: 52 - DNA, 24 bp, Artificial Sequence
  Description of Artificial Sequence Synthetic oligonucleotide
cacaagttagggtctcaggtatcg

SEQ ID NO: 53 - DNA, 22 bp, Artificial Sequence
  Description of Artificial Sequence Synthetic oligonucleotide
gttctgtagtgcactggtatcg

SEQ ID NO: 54 - DNA, 25 bp, Artificial Sequence
  Description of Artificial Sequence Synthetic oligonucleotide
tatgcaacctactacctctgtatcg

SEQ ID NO: 55 - DNA, 26 bp, Artificial Sequence
  Description of Artificial Sequence Synthetic oligonucleotide
ctgtacaaactactacctcagtatcg

SEQ ID NO: 56 - DNA, 27 bp, Artificial Sequence
  Description of Artificial Sequence Synthetic oligonucleotide
tgtaaaccatgatgtgctgctgtatcg

SEQ ID NO: 57 - DNA, 25 bp, Artificial Sequence
  Description of Artificial Sequence Synthetic oligonucleotide
ctgcactgtaagcacttttgtatcg

SEQ ID NO: 58 - DNA, 29 bp, Artificial Sequence
  Description of Artificial Sequence Synthetic oligonucleotide
tccataaagtaggaaacactacagtatcg

SEQ ID NO: 59 - DNA, 22 bp, Artificial Sequence
  Description of Artificial Sequence Synthetic oligonucleotide
ctgcttttgggattccgtatcg

SEQ ID NO: 60 - DNA, 29 bp, Artificial Sequence
  Description of Artificial Sequence Synthetic oligonucleotide
gctttgacaatactattgcactggtatcg

(19) United States
(12) Patent Application Publication
(10) Pub. No.: US 2016/0215328 A1
(43) Pub. Date: Jul. 28, 2016

(54) METHODS, COMPOSITIONS AND SYSTEMS FOR THE ANALYSIS OF NUCLEIC ACID MOLECULES

(71) Applicant: SOMAGENICS, INC., Santa Cruz, CA (US)

(72) Inventors: Sergei A. KAZAKOV, San Jose, CA (US); Anne DALLAS, Santa Cruz, CA (US); Heini ILVES, Santa Cruz, CA (US); Sumedha JAYASENA, Thousand Oaks, CA (US); Brian H. JOHNSTON, Scotts Valley, CA (US)

(21) Appl. No.: 14/771,195

(22) PCT Filed: Feb. 27, 2014
(86) PCT No.: PCT/US14/19055

§ 371 Date: Aug. 27, 2015

Related U.S. Application Data
(60) Provisional application No. 61/771,543, filed on Mar. 1, 2013.

(51) Int. Cl. C12Q 1/68

(52) U.S. Cl. CPC C12Q 1/6837

(57) ABSTRACT

Methods, systems and compositions are provided for analyzing one or more nucleic acid molecules. The methods, systems and compositions may comprise one or more target specific-oligonucleotide probes (TSPs). The TSPs may hybridize to nucleic acid molecules that are less than or equal to 200 nucleotides in length. The nucleic acid molecules may be small RNA molecules (e.g., miRNA, ncRNA, siRNA, shRNA). The methods, systems and compositions fmd use in a number of applications, for example, isolation of nucleic acid molecules, analysis of low abundance nucleic acid molecules, and/or enrichment of nucleic acid molecules.

### CROSS REFERENCE

This application claims the benefit of U.S. provisional application Ser. No. 61/771,543, filed Mar. 1, 2013; which is incorporated by reference in its entirety.

### FIELD OF THE INVENTION

The present invention is in the field of molecular diagnostics. More specifically, it concerns methods, systems and compositions useful for identification, detection, quantification, expression profiling and stabilizing of small RNAs, both naturally occurring and man-made. The present invention finds use in a variety of genomic research and diagnostic applications, in fields including medicine, agriculture, food, and biodefense. The RNA(s) of interest may represent biomarker(s) correlating to specific types of cancer or other diseases such as genetic and metabolic disorders and infectious disease.

### BACKGROUND OF THE INVENTION

The discovery of microRNAs (miRNAs) and other short RNAs such as small interfering RNAs (siRNA), and short non-coding RNAs (snRNA) has led to a rapid expansion of research elucidating their expression and diverse biological functions.

Recent studies have shown that distinct expression patterns of miRNAs are associated with specific types of cancer and certain other diseases, suggesting that miRNAs could represent a new class of biomarkers and prognostic indicators (Zhang and Farwell 2008). Good biomarkers can facilitate earlier diagnosis, which typically leads to better treatment outcomes. miRNAs also can be used as therapeutic agents as well as targets for antisense drugs (anti-miRs).

Knowledge of the absolute and relative expression levels (expression profiles) of miRNAs in a variety of biological and clinical samples can be useful for understanding the biogenesis of miRNAs, regulation of biochemical pathways by miRNAs, and identification of miRNA biomarkers.

Examples of current methods for detecting and/or quantifying small RNAs (e.g., miRNAs) include miRNA microarrays, BeadArray, Invader Assays, SYBR-based miRNA RT-qPCR assays, and Padlock probe-based assays. Stem-loop RT based TaqMan™ MicroRNA Assays may also be used to quantify and/or detect miRNAs. RT-qPCR-based methods are currently preferred for measuring levels of miRNAs (Kroh et al. 2010), but they are hampered by challenges associated with the requirement for isolating total RNA prior to analysis. The majority of blood-borne (circulating) miRNAs occur in complexes with lipids and/or proteins or are encapsulated within exosomes, all of which protect them from degradation by nucleases (Arroyo et al. 2011; Gallo et al. 2012) but can also hinder their detection. Without RNA isolation, these complexes, along with blood-borne nucleases and inhibitors of RT-PCR reactions like heparin (Kim et al. 2012a) can prevent detection or reduce the number of detectable miRNAs. Heparin is an endogenous component of blood and is commonly added as an anticoagulant during blood collection. In some cases the only plasma samples available may be heparinized (Kim et al. 2012a).

Most total RNA isolation methods require organic solvent extraction with subsequent ethanol precipitation or spin-column purification. These procedures result in reduced sensitivity and substantial sample-to-sample variability in both absolute and relative miRNA levels because of incomplete and inconsistent miRNA recovery, a consequence of the small size of miRNAs and the low concentrations at which they are typically found in plasma and serum (Etheridge et al. 2011; McDonald et al. 2011; Moltzahn et al. 2011; Kim et al. 2012a). Normalizing results to a synthetic miRNAs spike-in control does not solve this variability problem (McDonald et al. 2011). Also, a reliable, universal internal reference RNA (having the same concentration in different plasma or serum samples and thus usable for data normalization between different samples) has not been identified among the miRNAs present in plasma or serum (Etheridge et al. 2011; Reid et al. 2011; Zen and Zhang 2012).

Also, with standard RNA isolation procedures, it is difficult to eliminate RT-PCR inhibitors that co-purify with total RNA and, as a result, sensitivity cannot be increased by scaling up the quantity of RNA used in each sample assay (Kim et al. 2012a). In addition, the isolated total RNA or enriched small RNA fractions contain abundant fragments of unrelated RNAs (such as ribosomal RNA and tRNA); these can serve as primers for reverse transcription (RT) and PCR and can also compromise detection of low abundant miRNAs by specific primers added exogenously.

The high detection limit for typical RT-qPCR assays can also prevent the detection and/or quantification of low abundance RNAs. For example, the low concentrations of circulating miRNAs can be below the limit of detection for typical RT-qPCR assays (Kroh et al. 2010). In another example, among 190 miRNAs found in serum by deep sequencing, only 101 miRNAs could be validated/detected by RT-qPCR (Reid et al. 2011).

Thus, current RT-qPCR methods are often better suited to profiling moderately abundant circulating miRNAs that also exhibit relatively large differences in levels between normal and disease-associated samples rather than miRNAs that are present at relatively low levels in blood and/or exhibit small differences in expression between normal and disease-associated samples (Etheridge et al. 2011; McDonald et al. 2011; Moltzahn et al. 2011).

Hybridization methods that involve the direct capture of solution miRNAs onto a solid phase, including microarrays and bead-based assays, are used for miRNA quantification as alternatives to RT-qPCR (Zheng et al. 2011). However, these hybridization-based assays comprise target-specific capture probes immobilized to either target-designated surface locations or codes. Often, target-designated beads provide less sensitivity and less sequence-specificity than RT-qPCR.

So called “solid-phase” RT-PCR methods have been previously described for detection of mRNAs. Some of these methods use biotinylated oligonucleotide probes for capturing large RNAs such as mRNAs or viral RNAs from a solution onto a solid support coated by streptavidin to allow washing away of RT-PCR inhibitors along with unrelated nucleic acids and to permit the analysis of larger solution volumes (Yolken et al. 1991; Regan and Margolin 1997). Typically in these methods, mRNAs are captured at their polyadenylated 3′ ends on immobilized oligo(dT) probes, which then serve as reverse transcription (RT) primers for reverse transcription (Jost et al. 2007; Tanaka et al. 2009). Also, RT primers that bind to RNA molecules at sequences separate from those used to bind capture probes have been used for detection of viral RNAs (Regan and Margolin 1997; Mitsuhashi et al. 2006). The capture probes and RT-PCR primers used in latter assays were designed to be specific to distant segments of mRNAs to avoid any overlap and false-positive self-amplification. However, these “solid-phase” RT-PCR methods cannot be applied directly to small RNA molecules that have no poly(A)-tail and are nearly the same size as an ordinary RT or PCR primer, since two PCR primers are required for exponential amplification.

The methods, compositions and systems disclosed enable analysis of small RNAs (e.g. tasiRNA, piRNA, miRNA, and siRNA). The methods, compositions and systems disclosed herein may improve the sensitivity and/or accuracy of small RNA detection over current methods.

### SUMMARY OF THE INVENTION

Methods, compositions and systems are provided for the analysis of RNA molecules. The RNA molecules may be directly analyzed from samples. The samples may comprise a biological fluid, cell(s) or tissue, or lysates thereof. The RNA molecules may be analyzed without prior isolation of total RNA from the sample. The methods, compositions, and systems may exclude isolation of total RNA using organic solvents, ethanol precipitation or column-based procedures.

In some embodiments, the sample is a tissue lysate, a cell lysate, or an extracellular biological fluid (such as whole blood, plasma, serum, saliva, urine, sweat, sperm and breast milk) or the fluid lysate. In some embodiments, the sample is a lysate of formalin-fixed paraffin-embedded (FFPE) tissue blocks. In some embodiments, the sample is a crude nucleic acid extract.

The sample may be from a mammal, avian, amphibian, reptile, plant, bacteria, virus, or pathogen. The sample may comprise one or more cells derived from mammal, avian, amphibian, reptile, plant, bacteria or one or more pathogenically-infected cells. The mammal may be a human, goat, sheep, cow, pig, cat, dog, mouse, rat, or rabbit.

The samples may be from the same source. The samples may be from different sources. The samples may be from 2, 3, 4, 5, 6, 7, 8, 9, 10 or more different sources. The samples may be collected at the same time. The samples may be collected at different times. The samples may be collected at two or more different time points. The samples may be collected at 2, 3, 4, 5, 6, 7, 8, 9, 10 or more different time points.

Disclosed herein are methods and compositions for direct detection of RNA molecules without their chemical or enzymatic modification, labeling, or ligation of adapters to the RNA molecules, or extension of their ends prior to or after hybridization of a target-specific oligonucleotide probe to the RNA molecules.

In some embodiments, the RNA molecule is 200 or fewer nucleotides or base pairs in length. The RNA molecule may be 190 or fewer nucleotides or base pairs in length. The RNA molecule may be 180 or fewer nucleotides or base pairs in length. The RNA molecule may be 170 or fewer nucleotides or base pairs in length. The RNA molecule may be 160 or fewer nucleotides or base pairs in length. The RNA molecule may be 150 or fewer nucleotides or base pairs in length. The RNA molecule may be 140 or fewer nucleotides or base pairs in length. The RNA molecule may be 130 or fewer nucleotides or base pairs in length. The RNA molecule may be 120 or fewer nucleotides or base pairs in length. The RNA molecule may be 110 or fewer nucleotides or base pairs in length. The RNA molecule may be 100 or fewer nucleotides or base pairs in length. The RNA molecule may be 90 or fewer nucleotides or base pairs in length. The RNA molecule may be 80 or fewer nucleotides or base pairs in length. The RNA molecule may be 70 or fewer nucleotides or base pairs in length. The RNA molecule may be 60 or fewer nucleotides or base pairs in length. The RNA molecule may be 50 or fewer nucleotides or base pairs in length. The RNA molecule may be 40 or fewer nucleotides or base pairs in length. The RNA molecule may be 30 or fewer nucleotides or base pairs in length. The RNA molecule may be 20 or fewer nucleotides or base pairs in length. The RNA molecule may be 10 or fewer nucleotides or base pairs in length.

The RNA molecule may be a small non-coding RNAs (ncRNAs). The small ncRNA may be a microRNA (miRNA), small interfering RNA (siRNA), trans-acting siRNA (tasiRNA); repeat-associated siRNA (rasiRNA); small hairpin RNA (shRNA), piwi-interacting RNA (piRNA), small nuclear RNA (snRNA), small nucleolar RNA (snoRNA), scan RNA (scnRNA), transcription initiation RNA (tiRNA), small modulatory RNA (smRNA), tiny non-coding RNA (tncRNA), QDE-2 interacting RNA (qiRNA), precursor miRNA (pre-miRNA), or short bacterial ncRNAs.

In some embodiments, the RNA molecule is a microRNA (miRNA). The RNA molecule may be a pri-miRNA (e.g., initial transcript), pre-miRNA, or mature miRNA. The pre-miRNA may be about 80 or fewer nucleotides in length. The pre-miR may be about 70 or fewer nucleotides in length. The pre-miR may be about 65 or fewer nucleotides in length. The mature miRNA may be about 30 or fewer nucleotides in length. The mature miRNA may be about 25 or fewer nucleotides in length. The mature miRNA may be between about 10 to about 30 nucleotides in length. The mature miRNA may be between about 15 to about 28 nucleotides in length. The mature miRNA may be between about 19 to about 24 nucleotides in length.

The RNA molecule may be a fragment of larger coding (such as mRNA and genomic viral RNAs) or a fragment of a larger non-coding RNAs such as ribosomal RNA, tRNA, non-protein-coding RNA (npcRNA), non-messenger RNA, functional RNA (fRNA), long non-coding RNA (lncRNA, and primary miRNAs (pri-miRNAs).

In one aspect of the invention, methods are provided for procedures and compositions that dissociate or disrupt all protein and/or lipid complexes with RNA molecules while inhibiting ribonucleases that may degrade RNA molecules. Such compositions may include denaturing agents, detergents and proteases that can inhibit enzymes used in RT-PCR.

In another aspect of the invention, methods and compositions are provided for hybridization of the released small RNAs with target-specific oligonucleotide probes (TSPs) prior to their amplification by RT-PCR.

In some instances, TSP comprises a RNA-specific portion. The RNA-specific portion is complementary to at least a portion of the RNA molecule. The sequence of the RNA-specific portion may be at least about 5%, 10%, 20%, 30%, 40%, 50%, 60%, 70%, 80%, 85%, 90%, 95%, 97%, 99% or complementary to the sequence of the RNA molecule. The length of the RNA-specific portion may range from about 3 nucleotides up to the full length of the RNA molecule. The length of the RNA-specific portion may be at least about 3, 4, 5, 6, 7, 8, 9, 10, 11, 12, 13, 14, 15, 16, 17, 18, 19, 20, 21, 22, 23, 24 or more nucleotides. The length of the RNA-specific portion may be at least about 1, 2, 3, 4, 5, 6, 7, 8, 9, 10, 11, 12, 13, 14, 15, 16, 17, 18, 19, 20, 21 nucleotides less than the full length of the RNA molecule.

In some embodiments, the TSPs bind or capture different isoforms (forming mismatched/imperfect duplexes) and isomirs of the RNA molecule. The TSPs can bind to or capture at least about 2, 3, 4, 5, 6, 7, 8, 9, 10, 11, 12, 13, 14, 15, 16, 17, 18, 19, 20 or more different isoforms or isomirs of the RNA molecule. The TSPs can bind to or capture at least about 25, 30, 35, 40, 45, 50, 55, 60, 65, 70, 75, 80, 85, 90, 95, 100 or more different isoforms or isomirs of the RNA molecule.

The RNA-specific portion of the TSP may be fully or partially complementary to the RNA molecule. Hybridization of the TSP to the RNA molecule may comprise 5 or fewer mismatches. Hybridization of the TSP to the RNA molecule may comprise 4 or fewer mismatches. Hybridization of the TSP to the RNA molecule may comprise 3 or fewer mismatches. Hybridization of the TSP to the RNA molecule may comprise 2 or fewer mismatches. Hybridization of the TSP to the RNA molecule may comprise 1 or fewer mismatches. Hybridization of the TSP to the RNA molecule may comprise 0 mismatches.

In some embodiments, the TSP comprises DNA; RNA; a mix of DNA and RNA residues or their modified analogs such as 2′-OMe, or 2′-fluoro (2′-F), or phosphorothioate (PS), or locked nucleic acid (LNA), or abasic sites. In some embodiments, the TSP comprises one or more deoxyuracil (dU) residues that could be enzymatically cleaved by uracil-DNA-glycosylase. In some embodiments, the TSP comprises one or more modified nucleotide residues. The modified nucleotide residues can be cleaved chemically or enzymatically. The modified nucleotide residues may enable cleavage and/or degradation of the TSPs, while keeping the hybridized target RNA intact.

Disclosed herein are methods, compositions, and systems comprising one or more target-specific oligonucleotide probes (TSPs). The methods, compositions, and systems may comprise a plurality of TSPs. The plurality of TSPs may comprise 1, 2, 3, 4, 5, 6, 7, 8, 9, 10, 11, 12, 13, 14, 15, 16, 17, 18, 19, 20 or more TSPs. The plurality of TSPs may comprise 30, 40, 50, 60, 70, 80, 90, 100, 125, 150, 175, 200, 225, 250, 275, 300, 350, 400, 450, 500, 550, 600, 650, 700, 750, 800, 850, 900, 950, 1000 or more TSPs. The plurality of TSPs may comprise 2, 3, 4, 5, 6, 7, 8, 9, 10, 11, 12, 13, 14, 15, 16, 17, 18, 19, 20, 30, 40, 50, 60, 70, 80, 90, 100, 150, 200, 250 or more different TSPs. The plurality of TSPs may comprise multiple copies of one or more TSPs. The plurality of TSPs may comprise 2, 3, 4, 5, 6, 7, 8, 9, 10, 11, 12, 13, 14, 15, 16, 17, 18, 19, 20, 30, 40, 50, 60, 70, 80, 90, 100, 150, 200, 250 or more identical TSPs.

The method for analyzing one or more RNA molecules can comprise hybridizing one or more TSPs to one or more RNA molecules in a sample to form a TSP-hybridized RNA molecule. The method can comprise analyzing a plurality of RNA molecules. The plurality of RNA molecules may comprise 1, 2, 3, 4, 5, 6, 7, 8, 9, 10, 11, 12, 13, 14, 15, 16, 17, 18, 19, 20 or more RNA molecules. The plurality of RNA molecules may comprise 30, 40, 50, 60, 70, 80, 90, 100, 125, 150, 175, 200, 225, 250, 275, 300, 350, 400, 450, 500, 550, 600, 650, 700, 750, 800, 850, 900, 950, 1000 or more RNA molecules. The plurality of RNA molecules may comprise 2 or more different RNA molecules, 2 or more identical RNA molecules, or a combination thereof.

The method may comprise enrichment of the RNA molecule. The method may comprise concentrating the RNA molecule. The method may comprise separating the RNA molecule. Enrichment, concentration, and/or separation of the RNA molecules may comprise capture of the TSP-hybridized RNA molecule.

The method can comprise capture of the TSP-hybridized RNA molecule to produce one or more captured RNA molecules. The method can comprise consecutive capture of a plurality of TSP-hybridized RNA molecules to produce a plurality of captured RNA molecules. Capturing the TSP-hybridized RNA molecule(s) may comprise one or more solid supports. Capturing the TSP-hybridized RNA molecule(s) may comprise a magnetic separator.

The method can further comprise removing one or more solutes from sample. Removing the one or more solutes from the sample may comprise washing away other solutes.

The method may further comprise removal of the TSP from the TSP-hybridized RNA molecule to produce a non-hybridized RNA molecule.

The method may further comprise reverse transcribing the TSP-hybridized RNA molecule or derivative thereof to produce a TSP-cDNA molecule. The method may further comprise reverse transcribing the non-hybridized RNA molecule to produce a non-hybridized cDNA molecule.

The method may further comprise amplifying the TSP-hybridized RNA molecule or a derivative thereof (e.g., captured RNA molecule, TSP-cDNA molecule) to produce a TSP amplicon. The method may further comprise amplifying the non-hybridized RNA molecule or derivative thereof (e.g., non-hybridized cDNA molecule) to produce a non-hybridized amplicons.

The method may further comprise quantifying the RNA molecule. Quantifying the RNA molecule may comprise detection of the TSP-hybridized RNA molecule or a derivative thereof.

Derivatives of the TSP-hybridized molecule include, but are not limited to, the captured RNA molecule, cDNA molecule (e.g., TSP-cDNA molecule, non-hybridized cDNA molecule) or an amplicon of the RNA molecule (e.g., TSP amplicons, non-hybridized amplicons).

The method may further comprise hybridizing in solution the TSP-hybridized molecules to one or more solid supports. The one or more solid supports may be an array, microarray, bead, or a combination thereof.

The method may further comprise attaching the TSPs to one or more solid supports prior to hybridizing the TSPs to the RNA molecules. Attaching the TSPs to the solid support may comprise covalent attachment. Attaching the TSPs to the solid support may comprise non-covalent attachment. The TSPs may be hybridized to random locations on the solid support.

In some embodiments, the solid phase/support on which the TSP is immobilized is selected from the group comprising beads, membranes, filters, slides, arrays, microarrays, microtiter plates, and microcapillaries. In some embodiments, the immobilization is by a non-covalent interaction. In some such embodiments, the non-covalent interaction is mediated by an oligonucleotide and/or non-nucleotide linker.interaction. In some such embodiments, the TSP comprises a hapten group attached to either 5′- or 3′-ends ends of the TSP via non-nucleotide and/or oligonucleotide linkers; or a 5′- or 3′-end oligonucleotide linker complementary to capture oligonucleotides immobilized on the solid support. In certain embodiments, the hapten group is selected from biotin and digoxigenin. In embodiments in which the hapten is biotin, the solid support is coated with streptavidin or with antibodies specific for biotin. In embodiments in which the hapten is digoxigenin, the solid support is coated with antibodies specific for digoxigenin. In other embodiments, the immobilization is by a covalent linkage to the solid support. In some such embodiments, the covalent linkage is mediated by an oligonucleotide and/or non-nucleotide linker.

In some embodiments, both TSPs and primers bind to identical or overlapping sequences of the RNA molecule.

In some embodiments, the TSPs comprise unmodified deoxynucleotides whereas in other embodiments, these probes could contain modified nucleotides such as RNA, LNA, 2′-OMe, phosphorothioates and other residues known in the art that can increase or decrease the affinity of the probes for the small RNA molecules to provide the desired thermostability for the probe-RNA duplexes.

In some embodiments, the TSP comprises a blocking group at the 3′-end. The blocking group may prevent the enzymatic extension of the 3′ end, e.g., 3′-p, or 3′-amino, or 2′, 3′-dideoxy nucleoside (ddN), or 3′-inverted 3′-3′ deoxy nucleoside (idN). The blocking group may enable the TSP to to avoid interference with RT-PCR reactions and/or prevent false-positive amplification reactions in the RNA molecule. The blocking group may prevent the TSP from serving as a primer. The blocking group may prevent extension of the TSP.

In some embodiments, the TSP contains one or more residues that cannot be replicated by DNA polymerase. The one or more residues may be selected from the group comprising abasic site(s), nucleoside(s) with 2′-OMe or 2′-F modifications, or non-nucleotide linkers. The TSP may comprise an internal, stable hairpin. The TSP may comprise one or more features that prevent it from serving as a template for amplification.

In some embodiments, the TSP does not form a single-stranded overhang at the 5′ end of TSP when hybridized to the RNA molecule. In some instances, the TSP portion of the TSP-hybridized RNA molecule cannot serve as template for extension. The TSP portion may not serve as a template for RNA molecule 3′-end extension.

In some embodiments, the TSPs further comprise a linker. The linker may be a single-stranded overhang at the end of TSP. The linker may comprise a sequence that is non-complementary to RNA molecule or primers. The linker may comprise one or more nucleotides, non-nucleotides, or a combination thereof. The linker may be used to distance the RNA-specific portion of the TSP from the surface of the solid support. The linker may enable hybridization or attachment of the TSP to the solid support. The linker can improve the efficiency of hybridization between TSP and RNA molecule. The linker may comprise an anchor group or hapten. The linker may enable capture of the TSP or TSP-hybridized RNA molecule.

In some embodiments, TSPs are used to enrich for one or more RNA molecules. In some instances, enrichment of the one or more RNA molecules does not require strict sequence-specificity. Enrichment may comprise capture of various forms of an RNA molecule. For example, enrichment may comprise capture of various forms of a miRNA (e.g., pre-miRNAs, miRNA isomirs, and miRNA isoforms). miRNA isoforms may differ by single nucleotide polymorphisms. miRNA isomirs may differ by nucleotide additions or deletions at one or more ends. These various forms can then be individually quantified using RT-qPCR assays specific to each form of RNA molecules. Capture of the various forms of RNA molecules may occur simultaneously. Capture of the various forms of RNA molecules may occur sequentially. Capture of the various forms of RNA molecules may occur in the same solution. The various forms of RNA molecules may be captured fluidically.

Further disclosed herein are methods for separating one or more small RNA molecules from a sample comprising a plurality of nucleic acid molecules. The plurality of nucleic acid molecules may comprise mRNAs, ribosomal RNA, tRNAs, genomic DNA, DNA, DNA fragments, RNA fragments, small RNAs, or a combination thereof. The sample may comprise a mixture of nucleic acid molecules. The sample may further comprise one or more peptides or polypeptides. The small RNAs may comprise target small RNAs (e.g., small RNAs of interest) and non-target small RNAs. The method may comprise (a) capturing one or more RNA molecules in a sample with one or more TSPs to produce a first subset comprising one or more TSP-hybridized RNA molecules and a second subset comprising non-hybridized molecules; and (b) removing non-hybridized molecules. The first subset and the second subset may be fluidically separated. The one or more RNA molecules may be 200 or fewer nucleotides in length. The one or more RNA molecules may be 100 or fewer nucleotides in length. The one or more RNA molecules may be 50 or fewer nucleotides in length.

In some instances, the methods further comprise reverse transcribing at least a portion of the RNA molecule portion of the TSP-hybridized RNA molecule to produce a complementary DNA (cDNA) molecule.

The methods may further comprise releasing the one or more RNA molecules from the one or more TSP-hybridized RNA molecules. Releasing the one or more RNA molecules may comprise dissociation of the TSP-hybridized RNA molecules to produce a released RNA molecule. Dissociation may comprise subjecting the TSP-hybridized RNA molecules to one or more denaturing conditions. The one or more denaturing conditions may comprise a change in temperature, change in pH, addition of one or more enzymes, change in solutions/buffers, or any combination thereof. For example, denaturing may comprise incubating the TSP-hybridized RNA molecules in low salt conditions at high temperature (e.g., by washing with deionized water or 0.1 mM EDTA at greater than or equal to 75° C.). Alternatively, the denaturing conditions may comprise a temperature greater than or equal to 50° C. The denaturing conditions may comprise a temperature greater than or equal to 55° C. The denaturing conditions may comprise a temperature greater than or equal to 60° C. The denaturing conditions may comprise a temperature greater than or equal to 65° C. The denaturing conditions may comprise a temperature greater than or equal to 70° C. The denaturing conditions may comprise a temperature greater than or equal to 80° C. The denaturing conditions may comprise a temperature greater than or equal to 85° C. The denaturing conditions may comprise a temperature greater than or equal to 90° C. The denaturing conditions may comprise a temperature greater than or equal to 95° C. The denaturing conditions may comprise a temperature greater than or equal to 100° C.

The method may further comprise separating the denatured RNA molecules from the TSPs. The denatured RNA molecules may be fluidically separated from the TSPs. Alternatively, or additionally, the denatured RNA molecules are physically separated from the TSPs. Separating the denatured RNA molecules from the TSPs may comprise transferring the denatured RNA molecules to one or more containers. The one or more containers may be a tube (e.g., Eppendorf tube, centrifuge tube, PCR tube), well (e.g., microtiter well), plate, etc.

The methods may further comprise detecting the TSP-hybridized RNA molecule or a derivative thereof (e.g., denatured RNA molecule, amplicon, etc). Detecting may comprise an RT-qPCR method (e.g., by the TaqMan micro RNA assay described by Chen et al. 2005).

In some instances, the TSP-hybridized RNA molecules or derivative thereof are circularized to produce a circularized RNA molecule or derivative thereof (e.g., circularized cDNA, circularized amplicons, etc). Circularization may occur without prior dissociation and/or release of the TSP-hybridized RNA molecules. Alternatively, or additionally, circularization may occur with the release/dissociation of the RNA molecule from TSP-hybridized RNA molecule. Circularization may occur prior to detection of the TSP-hybridized RNA molecule or derivative thereof. Circularization may occur prior to amplification of the TSP-hybridized RNA molecule or derivative thereof. Circularization may occur after dissociation and/or release of the TSP-hybridized RNA molecule or derivative thereof. Circularization may occur after reverse transcription of the TSP-hybridized RNA molecule or derivative thereof.

In other embodiments of this invention, the circularization of the TSP-hybridized RNA molecules or derivative thereof is carried out by one or more ligases. The ligases may be thermostable ligases. The circularization may occur at a temperature that is higher than the melting temperature (Tₘ) of the TSP-hybridized RNA molecule. The circularization may occur at 50° C. or greater. The circularization may occur at 55° C. or greater. The circularization may occur at 60° C. or greater. The circularization may occur at 65° C. or greater. The one or more ligases may be a RNA ligase, DNA ligase, RNA/DNA ligase, or a combination thereof. The one or more ligases may ligate a single-stranded DNA molecule (e.g., cDNA molecule). The one or more ligases may ligate single-stranded DNA molecules in the absence of complementary ends. Alternatively, the one or more ligases may ligate DNA ends that are annealed adjacent to each other on a complementary DNA sequence. The thermostable ligase may be CircLigase or CircLigase II (from Epicentre), or Thermostable RNA ligase (from Epicentre), or Thermostable 5′ AppDNA/RNA Ligase (from New England Biolabs). The one or more ligases may be a T4 DNA Ligase and/or Ampligase® DNA Ligase.

The methods may further comprise reverse transcribing the circularized RNA molecules to produce a cDNA product. The cDNA product may be a multimeric cDNA product, which comprises multiple cDNA copies of the RNA molecule. Reverse transcription may comprise rolling circle amplification mechanism (RT-RCA).

The method may further comprise quantification of the cDNA molecules. The cDNA molecules may be quantified by qPCR using miR-ID method (Kumar et al. 2011).

Further disclosed herein is a method for analyzing one or more RNA molecules in a sample comprising (a) hybridizing one or more RNA molecules in a sample with one or more TSPs to produce one or more TSP-hybridized RNA molecules; and (b) circularizing the one or more RNA molecules to produce a circularized RNA molecule, wherein the one or more RNA molecules were hybridized to the one or more TSPs. Circularizing the one or more RNA molecules may comprise incubating the TSP-hybridized RNA molecules at 50° C. or greater to dissociate the TSP-hybridized RNA molecule to produce one or more free/released RNA molecules. Circularizing the one or more RNA molecules may further comprise contacting the free RNA molecules with one or more ligases. The one or more ligases may be thermostable ligases. The one or more ligases may be CircLigase. The method may further comprise reverse transcribing the circularized RNA molecule to produce one or more cDNA molecules. The one or more cDNA molecules may be a multimeric cDNA molecule, wherein the multimeric cDNA molecule comprises two or more copies of the circularized RNA molecule. The method may further comprise quantifying the cDNA molecule by qPCR. qPCR may be performed using the miR-ID method disclosed in Kumar et al (2011). The method may provide higher sensitivity (or signal-to-noise ratio) than use of the TaqMan microRNA detection on linear RNA. Detection of the RNA molecules may be at least about 1.5, 1.75, 2, 2.25, 2.5, 2.75, 3, 3.25, 3.5, 3.75, 4, 4.25, 4.5, 4.75, 5, 5.25, 5.5, 5.75, 6, 7, 8, 16, 32, 64, 128, 256, 512, 1024-fold or greater more sensitive than detection by current methods (e.g., TaqMan).

The methods may further comprise quantitatively detecting the PCR products by real-time qPCR. Detection may comprise using TaqMan or similar probes. The methods, compositions and systems may further comprise one or more DNA polymerases. The DNA polymerases may comprise exonuclease activity. The primers or probes may induce signals upon degradation by DNA polymerase with 5′-exonuclease activity. In other embodiments, real-time PCR is performed using either a single dye such as SYBR Green or EvaGreen dyes.

In some embodiments, the RNA molecule comprises a 5′ end that comprises a 5′-phospate (5′-p); a 5′-hydroxyl (5′-OH); a 5′-cap; or a 5′-triphosphate (5′-ppp). The method may further comprise converting the 5′ end to a 5′-phosphate prior to circularization of RNA molecule. Conversion of the 5′ end may comprise enzymatic conversion.

In some embodiments, the RNA molecule comprises a 3′ end that comprises a 3′-hydroxyl (3′-OH); a 3′-phospate (3′-p); or a 2′, 3′-cyclic phosphate (2′, 3′>p). The method may further comprise converting the 3′ end to a 3′-OH prior to circularization of RNA molecule.

In some embodiments, the RNA molecule comprises a 2′ group at the 3′ end selected from a 2′-OH or a 2′-oxymethyl (2′-OMe).

In some instances, methods are provided for detecting and quantifying one or more RNA molecules in a sample by using one or more TSPs. In some instances, quantifying the one or more RNA molecules comprises hybridizing a plurality of hybridized to one or more RNA molecules simultaneously in a multiplex format.

In some instances, the provided methods decrease or eliminate the loss of small RNAs that usually occurs under standard total RNA isolation conditions, reduce the number of steps for preparation of miRNAs for RT-qPCR analysis to streamline the procedure, minimize the variability of RNA recovery from different samples, and/or reduce the RNA recovery time and facilitate its automation. In some instances, the provided methods increase the accuracy in determining absolute RNA copy numbers, and/or enable expression profiling of small RNAs of interest (target RNAs) while removing inhibitors of amplification reactions and depleting irrelevant small RNAs, including degradation products of larger RNAs or DNA. The latter features increase the efficiency of amplification of specific RNA sequences and reduce background amplification of non-specific sequences, thereby increasing the sensitivity (signal-to-noise ratio) and multiplexing capability of RT-PCR.

Disclosed herein is a method for analyzing one or more nucleic acid molecules, comprising (a) contacting one or more samples comprising one or more nucleic acid molecules with one or more target-specific oligonucleotide probes (TSPs) to produce one or more TSP-hybridized nucleic acid molecules, wherein (i) the one or more TSPs hybridize to one or more nucleic acid molecules that may be 200 or fewer nucleotides or base pairs in length; (ii) the one or more TSPs may comprise a nucleic acid-specific portion that hybridizes to at least a portion of the one or more nucleic acid molecules; and (iii) the length of the nucleic acid-specific portion may be less than or equal to the full length of the nucleic acid molecule; (b) capturing the TSP-hybridized nucleic acids on a solid support to produce captured nucleic acid molecules; (c) removing one or more analytes and other solution components from the sample, wherein the one or more analytes may be not hybridized to the one or more TSPs; (d) releasing the captured nucleic acid molecules into solution to produce released nucleic acid molecules; and (e) detecting the released nucleic acid nucleic acid molecules.

The method may further comprise releasing one or more nucleic acid molecules from a complex prior to contacting the one or more samples with one or more TSPs. Removing the one or more analytes from the sample may comprise washing the one or more captured TSP-hybridized nucleic acid molecules. Releasing the captured nucleic acid molecules into solution may comprise dissociating/releasing one or more nucleic acid molecules from the one or more TSP-hybridized nucleic acid molecules to produce one or more non-hybridized/free nucleic acid molecules, wherein the non-hybridized nucleic acid molecules may be nucleic acid molecules that may be no longer hybridized to the one or more TSPs.

The one or more nucleic acid molecules may be separated from the one or more TSP-hybridized molecules in a solution. The one or more non-hybridized nucleic acid molecules may be fluidically separated from the one or more TSPs.

Detecting the one or more TSP-hybridized nucleic acid molecules may comprise detecting the one or more captured TSP-hybridized nucleic acid molecules. Detecting the one or more TSP-hybridized nucleic acid molecules may comprise detecting the one or more non-hybridized nucleic acid molecules.

Detecting the one or more TSP-hybridized nucleic acid molecules may comprise circularizing the one or more TSP-hybridized nucleic acid molecules or derivative thereof to produce one or more circularized nucleic acid molecules. The one or more TSP-hybridized molecules may be circularized in a multiplex reaction. Two or more TSP-hybridized nucleic acid molecules or derivatives thereof may be circularized. Five or more TSP-hybridized nucleic acid molecules or derivatives thereof may be circularized. Ten or more TSP-hybridized nucleic acid molecules or derivatives thereof may be circularized. Fifteen or more TSP-hybridized nucleic acid molecules or derivatives thereof may be circularized. Twenty or more TSP-hybridized nucleic acid molecules or derivatives thereof may be circularized. Thirty or more TSP-hybridized nucleic acid molecules or derivatives thereof may be circularized. Forty or more TSP-hybridized nucleic acid molecules or derivatives thereof may be circularized.

Circularizing may occur without complete dissociation of TSP-hybridized nucleic acid molecules or derivatives. Circularization may occur with partial dissociation of TSP-hybridized nucleic acid molecules or derivatives.

Circularizing may comprise use of one or more ligases.Circularizing may comprise use of one or more thermostable ligases. The thermostable ligase may be a RNA ligase. The thermostable ligase may be a DNA ligase. The thermostable ligase may be a DNA/RNA ligase that can circularize both DNA and RNA molecules. The thermostable ligase may be a CircLigase.

The ends of the one or more TSP-hybridized molecules may overlap. In some instances, the ends of the one or more TSP-hybridized molecules do not overlap.

Circularizing may comprise heating the sample to a temperature greater than the melting temperature (Tm) of the one or more TSPs. The temperature may be greater than or equal to 50° C. The temperature may be greater than or equal to 55° C. The temperature may be greater than or equal to 60° C. The temperature may be greater than or equal to 65° C. The temperature may be greater than or equal to 67° C. The temperature may be greater than or equal to 70° C. The temperature may be greater than or equal to 72° C. The temperature may be greater than or equal to 75° C. The temperature may be greater than or equal to 77° C. The temperature may be greater than or equal to 80° C.

Detecting the one or more TSP-hybridized nucleic acid molecules may comprise reverse transcribing the one or more TSP-hybridized nucleic acid molecules or derivative thereof to produce one or more nucleic acid copy molecules, wherein the one or more nucleic acid copy molecules may be copies of the one or more TSP-hybridized nucleic acid molecules or a derivative thereof. The one or more TSP-hybridized nucleic acid molecules may be reverse transcribed in a multiplex reaction. Two or more TSP-hybridized nucleic acid molecules or derivatives thereof may be reverse transcribed. Five or more TSP-hybridized nucleic acid molecules or derivatives thereof may be reverse transcribed. ten or more TSP-hybridized nucleic acid molecules or derivatives thereof may be reverse transcribed. Fifteen or more TSP-hybridized nucleic acid molecules or derivatives thereof may be reverse transcribed. Twenty or more TSP-hybridized nucleic acid molecules or derivatives thereof may be reverse transcribed. Thirty or more TSP-hybridized nucleic acid molecules or derivatives thereof may be reverse transcribed. Forty or more TSP-hybridized nucleic acid molecules or derivatives thereof may be reverse transcribed.

Detecting the one or more TSP-hybridized nucleic acid molecules may comprise real-time RT-qPCR (qRT-PCR).

The length of the one or more primers may be less than or equal to about 12 nucleotides. The length of the one or more primers may be less than or equal to about 10 nucleotides. The length of the one or more primers may be less than or equal to about 9 nucleotides. The length of the one or more primers may be less than or equal to about 8 nucleotides. The length of the one or more primers may be less than or equal to about 7 nucleotides. The length of the one or more primers may be less than or equal to about 6 nucleotides. The length of the one or more primers may be between about 3 to to about 22 nucleotides.

The length of the one or more RT primers may be less than or equal to about 13 nucleotides. The length of the one or more RT primers may be less than or equal to about 11 nucleotides. The length of the one or more RT primers may be less than or equal to about 9 nucleotides. The length of the one or more RT primers may be less than or equal to about 7 nucleotides. The length of the one or more primers may be less than or equal to about 5 nucleotides. The length of the one or more RT primers may be less than or equal to about 4 nucleotides. The length of the one or more RT primers may be between about 4 to about 13 nucleotides.

The PCR primers may comprise one or more 5′-overlapping primer pairs. The length of the one or more PCR primers may be equal to about 15 nucleotides or longer. The length of the one or more PCR primers may be equal to about 17 nucleotides or longer. The length of the one or more PCR primers may be equal to about 19 nucleotides or longer. The length of the one or more PCR primers may be equal to about 21 nucleotides or longer. The length of the one or more PCR primers may be between about 13 to about 25 nucleotides.

The one or more samples may be biological samples. The one or more samples may be from one or more cells, tissues, fluids, secretions, excretions, or a combination thereof. The one or more samples may be from one or more lysates, fluids, extracellular fluids, nucleic acid extracts, nucleic acid extracts, purified nucleic acid samples, purified nucleic acid samples, subsets of one or more nucleic acid samples, or a combination thereof. The one or more samples may be from one or more cell lysates. The one or more fluids may comprise secretions, sweat, tears, saliva, spinal fluid, blood, plasma, serum, ocular fluid, urine, or a combination thereof. The one or more samples may be from one or more mammals. The one or more mammals may be selected from the group comprising humans, apes, goats, sheeps, dogs, cows, mice, rats, cats, pigs, horses, or a combination thereof. The one or more samples may be from one or more humans. The one or more nucleic acid molecules may be detected without prior extraction and/or purification of the one or more nucleic acid molecules.

The method may further comprise release of the one or more nucleic acid molecules from one or more protective complexes. The one or more protective complexes may be selected from the group comprising cells, circulating cells, exosomes, lipid vehicles, lipo-protein vehicles or protein complexes.

The one or more solid supports may be selected from the group comprising beads, membranes, filters, slides, arrays, microarrays, chips, microtiter plates, and microcapillaries. The bead may comprise a coated bead, magnetic bead, antibody-conjugated bead, or any combination thereof. The bead may be a streptavidin-coated magnetic bead.

The one or more TSPs further may comprise a linker. The linker may comprise a hapten. The hapten may be biotin.

Detecting the one or more TSP-hybridized nucleic acid molecules or derivative thereof may comprise amplifying the one or more TSP-hybridized nucleic acid molecules or derivative thereof to produce one or more amplicons. The derivative of the one or more TSP-hybridized nucleic acid molecules may be selected from the group comprising a captured TSP-hybridized nucleic acid molecule, non-hybridized nucleic acid molecule, circularized nucleic acid molecule, nucleic acid copy molecules, amplicons or a combination thereof.

The one or more nucleic acid molecules that may be hybridized to the one or more TSPs may comprise one or more RNA molecules. The one or more RNA molecules may comprise one or more microRNA (miRNA) molecules. The one or more RNA molecules may comprise one or more pre-miRNA, mature miRNA, or a combination thereof. The one or more nucleic acid molecules that may be hybridized to the one or more TSPs may comprise fragments of a DNA molecule, RNA molecule, or a combination thereof.

The one or more TSPs hybridize to one or more nucleic acid molecules that may be 150 or fewer nucleotides or base pairs in length. The one or more TSPs hybridize to one or more nucleic acid molecules that may be 100 or fewer nucleotides or base pairs in length. The one or more TSPs hybridize to one or more nucleic acid molecules that may be 70 or fewer nucleotides or base pairs in length. The one or more TSPs hybridize to one or more nucleic acid molecules that may be 50 or fewer nucleotides or base pairs in length. The one or more TSPs hybridize to one or more nucleic acid molecules that may be 40 or fewer nucleotides or base pairs in length.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Schematic of the miR-Direct approach.

FIG. 2. Direct quantification of miRNAs in plasma without capture with TagMan® (dark gray) or miR-ID® (light gray) detection (as described in Example 1).

FIG. 3A-B. Quantification of miRNAs in different volumes of plasma by miR-Direct using miR-ID detection (as described in Example 2). Two different sets of miRNAs were assayed either in 25, 100 and 400 μl (FIG. 3A) or in 50 and 400 μl (FIG. 3B) of plasma samples, respectively. The same amount of spike-in control miRNA (cel-39) was added to each plasma volume. The sensitivity of the assay for circulating miRNAs increases proportionally to the plasma input volume.

FIG. 4A-D. Quantification of miRNAs in various plasma samples by miR-Direct using miR-ID detection (as described in Example 3). FIG. 4A quantification of miR-16 by miR-Direct using miR-ID detection. FIG. 4B quantification of mir-125b by miR-Direct using miR-ID detection. FIG. 4C quantification of miR-148a by miR-Direct using miR-ID detection. FIG. 4D quantification of cel-39 (spike-in) by miR-Direct using miR-ID detection.

FIG. 5A-D. Quantification of miRNAs in various plasma samples using total RNA isolated from plasma by column purification with miR-ID detection (as described in Example 4). FIG. 5A quantification of miR-16 in various plasma samples using total RNA isolated from plasma by column purification with miR-ID detection. FIG. 5B quantification of miR-125b in various plasma samples using total RNA isolated from plasma by column purification with miR-ID detection. FIG. 5C quantification of miR-148a in various plasma samples using total RNA isolated from plasma by column purification with miR-ID detection. FIG. 5D quantification of cel-39 (spike-in) in various plasma samples using total RNA isolated from plasma by column purification with miR-ID detection.

FIG. 6A-D. Quantification of miRNAs in various plasma samples using total RNA isolated from plasma by column purification with TaqMan detection (as described in Example 5). FIG. 6A quantification of miR-16 in various plasma samples using total RNA isolated from plasma by column purification with TaqMan detection. FIG. 6B quantification of miR-125b in various plasma samples using total RNA isolated from plasma by column purification with TaqMan detection. FIG. 6C quantification of miR-148a in various plasma samples using total RNA isolated from plasma by column purification with TaqMan detection. FIG. 6D quantification of cel-39 (spike-in) in various plasma samples using total RNA isolated from plasma by column purification with TaqMan detection.

FIG. 7A-D. Quantification of miRNAs in plasma by miR-Direct using various miRNA elution conditions with TaqMan detection (as described in Example 6). FIG. 7A quantification of miRNAs in plasma by miR-Direct using miRNA elution conditions at 60° C. with TaqMan detection. FIG. 7B quantification of miRNAs in plasma by miR-Direct using miRNA elution conditions at 75° C. with TaqMan detection. FIG. 7C quantification of miRNAs in plasma by miR-Direct using miRNA elution conditions at 95° C. with TaqMan detection. FIG. 7D comparision of the amount of the detected miRNAs eluted at 60° C., 75° C. and 95° C.

FIG. 8. Quantification of miRNAs in 200-μl aliquots of a single plasma sample with miR-ID detection (as described in Example 2) using various times (0 or 30 minutes) of recovery of the captured miRNAs from the beads after miRNA dissociation and circularization.

FIG. 9. Quantification of circulating miRNAs (miR-16 and miR-106a) and spiked-in cel-miR-39 with miR-ID RT-qPCR assay after processing by miR-Direct (as described in Example 2) from 200-μl aliquots of plasma samples from a single individual that were collected into either heparin- or EDTA-containing tubes. The average of 3 replicates was plotted with error bars showing the standard deviation of the mean. There was no appreciable difference in levels of miRNAs detected in heparin vs. EDTA samples despite the fact that heparin is known to be a strong inhibitor of conventional RT-PCR assays.

### DETAILED DESCRIPTION OF THE INVENTION

Before the present methods and compositions are described, it is to be understood that this invention is not limited to any particular method or composition described, as such may, of course, vary. It is also to be understood that the terminology used herein is for the purpose of describing particular embodiments only, and is not intended to be limiting, since the scope of the present invention will be limited only by the appended claims. Examples are put forth so as to provide those of ordinary skill in the art with a complete disclosure and description of how to make and use the present invention, and are not intended to limit the scope of what the inventors regard as their invention, nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. The section headings used herein are for organizational purposes only and are not to be construed as limiting the subject matter described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order, which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The methods, compositions, and systems disclosed herein find use in a number of applications. For example, the methods, compositions and systems disclosed herein can be used for the detection, quantification, enrichment, and/or sequencing of RNA molecules. The RNA molecules may be detected and/or quantified by any method (e.g., amplification, hybridization, RT-PCR).

In addition, the methods, compositions and systems disclosed herein can be used in the construction of libraries. The libraries may comprise one or more RNA molecules. The libraries may comprise one or more small RNA molecules. The libraries may comprise fragments of large RNAs. These and other objects, advantages, and features of the invention will become apparent to those persons skilled in the art upon reading the details of the compositions and methods as more fully described herein.

Disclosed herein are methods of analyzing one or more RNA molecules. The methods generally comprise hybridizing one or more TSPs to one or more RNA molecules to form a TSP-hybridized RNA molecule. The RNA molecules can comprise small RNA molecules. The small RNAs may be equal to or less than about 300, 275, 250, 225, 200, 190, 180, 170, 160, 150, 140, 130, 120, 110, 100 or fewer nucleotides or base pairs in length. The small RNAs may be equal to or less than about 200 nucleotides or base pairs in length. The small RNAs may be less than about 150 nucleotides or base pairs in length. The small RNAs may be equal to or less than about 100 nucleotides in length. The small RNAs may be equal to or less than about 100, 90, 80, 70, 60, 50, 40, 30, 25 or fewer nucleotides or base pairs in length. In some instances, the methods further comprise reverse transcribing at least a portion of the RNA molecule portion of the TSP-hybridized RNA molecule to produce a cDNA copy of RNA molecule template. Alternatively, or additionally, the methods further comprise amplifying the TSP-hybridized RNA molecule to produce an RNA amplicon. The methods disclosed herein can further comprise isolating a TSP-hybridized RNA molecule to produce an isolated RNA molecule. The methods disclosed herein can further comprise quantifying the RNA molecule by detecting the TSP-hybridized RNA molecule and/or a derivative thereof (e.g., cDNA molecule, amplified RNA molecule). Detection of the TSP-hybridized RNA molecule may be by RT-qPCR. In other instances, the methods further comprise sequencing the TSP-hybridized RNA molecule and/or a derivative thereof (e.g., amplified RNA molecule, cDNA RNA molecule, isolated RNA molecule). Reverse transcribing, amplifying, and/or sequencing the TSP-hybridized RNA molecule may comprise hybridizing a primer to the TSP-hybridized RNA molecule. The primer may be equal to or less than about 30, 29, 28, 27, 26, 25, 24, 23, 22, 21, 20, 17, 15, 12, 11, 10, 9, 8, 7, 6, 5, 4, or 3 nucleotides in length. The primer may be between about 3 to about 8 nucleotides in length. The primer may be about 6 nucleotides in length. The primer may be about 5 nucleotides in length. The primer may be complementary to at least a portion of the RNA molecule. The primer may be complementary to at least about 3, 4, 5 or more nucleotides of the RNA molecule.

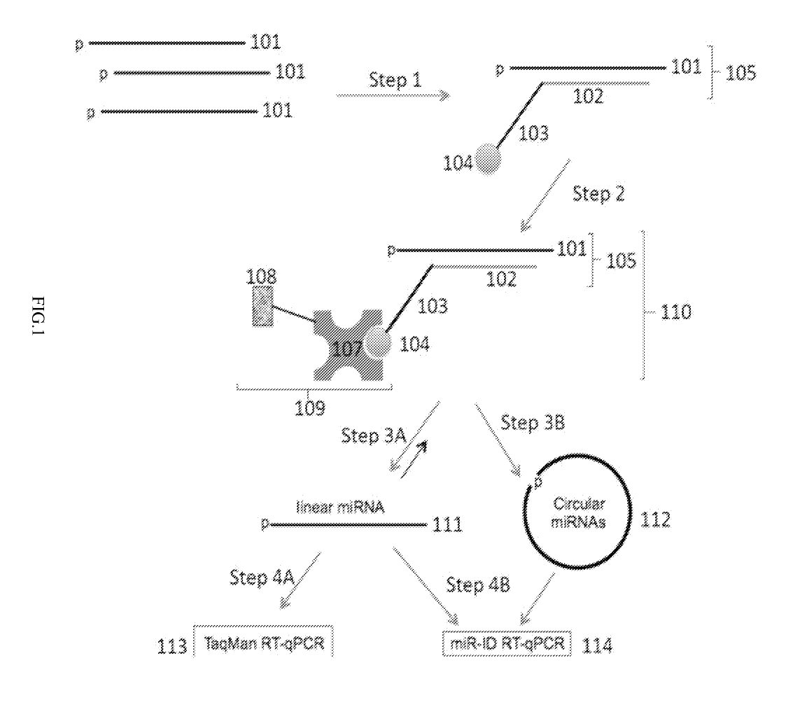

FIG. 1 shows a schematic of one method of analyzing one or more miRNA molecules. The methods for analyzing the one or more RNA molecules may comprise one or more steps as shown in FIG. 1. As shown in Step 1 of FIG. 1, the method may comprise hybridizing a RNA molecule (101) to a TSP (102) to form a TSP-hybridized RNA molecule (105). The RNA molecule may be a miRNA or anther small RNA. The TSP (102) may be linked to a linker (103). The linker (103) may be conjugated to a hapten (104). The hapten may be biotin. As shown in Step 2 of FIG. 1, the method may further comprise capturing the TSP-hybridized RNA molecule (105) to a solid support (109) to produce a captured TSP-hybridized RNA molecule (110). The hapten (104) may enable capturing of the TSP-hybridized RNA molecule (105) to the solid support (109). For example, the hapten (104) may be biotin and the solid support (109) may comprise streptavidin (107) conjugated to a bead (108). The method may comprise removal or separation of the non-conjugated molecules (106) from the TSP-hybridized RNA molecule (105). As shown in Step 3A of FIG. 1, the method may further comprise release/dissociation of the RNA molecule (101) from the captured TSP-hybridized molecule (110) to produce a released RNA molecule (111). As shown in Step 3A of FIG. 1, the released RNA molecule (111) may be linear. As shown in Step 4A and 4B, the released RNA molecule (111) may be further analyzed by methods including, but not limited to, TaqMan RT-qPCR (Step 5A), or miR-ID. As shown in Step 3B of FIG. 1, release of the RNA molecule (101) from the captured TSP-hybridized RNA molecule (110) may comprise circularization of the released RNA molecule to produce a circularized released RNA molecule (112). As shown in Step 4B, the circularized released RNA molecule (112) may be further analyzed by methods including, but not limited to miR-ID RT-qPCR. The method may further comprise dissociating of the RNA molecules from a protective complexes (e.g., cells, tissue, exosomes, lipid and/or protein complexes etc) and their release into solution prior to hybridization to the TSP. The method may further comprise analyzing the released RNA molecules by sequencing (111, 112). The method may further comprise quantifying the released RNA molecules (111, 112). The method may comprise (a) releasing miRNAs from their complexes with lipids and proteins in plasma; (b) hybridizing the released miRNAs with biotinylated target-specific oligonucleotide probes (biotinylated TSPs) in solution to produce TSP-hybridized miRNA molecules; (c) capturing the TSP-hybridized miRNA molecules on streptavidin-coated magnetic beads to produce captured miRNAs; (d) removing one or more non-captured molecules, wherein the non-captured molecules are not the captured miRNAs; (e) releasing the captured miRNAs from the beads into solution to produce free miRNAs; and/or (f) detection of the free miRNAs. Detection of the free miRNAs may comprise direct detection of the free miRNAs by the TaqMan microRNA RT-qPCR method, wherein the free miRNAs are linear. Alternatively, detection of the free miRNAs may comprise circularization of the free linear miRNAs to produce one or more circularized miRNAs, and quantification of circularized miRNAs by the miR-ID assay (Kumar et al. 2011). The method may further comprise amplifying the TSP-hybridized miRNA or a derivative thereof.

Further disclosed herein are compositions or systems for analyzing one or more RNA molecules. The compositions or systems may comprise one or more target-specific oligonucleotide probes (TSPs). The compositions or systems may further comprise one or more enzymes. The one or more enzymes may comprise a reverse transcriptase, polymerase, helicase, RNAse, or any combination thereof. The compositions and/or systems may further comprise a thermal cycler, sequencer, hybridization chamber, separator, magnetic separator, array, microarray, solid support, bead, or any combination thereof.

In other instances, the methods, systems, and kits disclosed herein can be used to amplify an RNA molecule. The methods generally comprise: (a) hybridizing one or more TSPs to a RNA molecule to produce a TSP-hybridized RNA molecule, wherein the RNA molecule is equal to or less than 200 nucleotides in length; (b) releasing the RNA molecule from the TSP-hybridized RNA molecule into solution to produce a released RNA molecule; and (c) amplifying the released RNA molecule or a derivative thereof. Amplifying the RNA molecule may comprise hybridizing a primer to the RNA molecule. The primer may be equal or less than about 30, 29, 28, 27, 26, 25, 24, 23, 22, 21, 20, 17, 15, 12, 11, 10, 9, 8, 7, 6, 5, 4, or 3 nucleotides in length. The primer may be between about 3 to about 8 nucleotides in length. The primer may be about 6 nucleotides in length. The primer may be about 5 nucleotides in length. The primer may be complementary to at least a portion of the RNA molecule. The primer may be complementary to at least about 3, 4, 5 or more nucleotides of the RNA molecule.

In other instances, the methods, systems, and kits disclosed herein can be used to reverse transcribe a RNA molecule. Generally, the methods, compositions, and kits comprise: (a) hybridizing one or more TSPs to a RNA molecule to produce a TSP-hybridized RNA molecule; (b) releasing the RNA molecule from the TSP-hybridized RNA molecule into solution to produce a released RNA molecule; and (c) reverse transcribing the released RNA molecule or a derivative thereof. Reverse transcribing the released RNA molecule may comprise hybridizing a primer to the TSP-hybridized RNA molecule. The primer may be equal or less than about 30, 29, 28, 27, 26, 25, 24, 23, 22, 21, 20, 17, 15, 12, 11, 10, 9, 8, 7, 6, 5, 4, or 3 nucleotides in length. The primer may be between about 3 to about 8 nucleotides in length. The primer may be about 6 nucleotides in length. The primer may be about 5 nucleotides in length. The primer may be complementary to at least a portion of the RNA molecule. The primer may be complementary to at least about 3, 4, 5 or more nucleotides of the RNA molecule.

In other instances, the methods, systems, and kits disclosed herein can be used to sequence a RNA molecule. The methods, compositions, and kits generally comprise: (a) hybridizing one or more TSPs to a RNA molecule to produce a TSP-hybridized RNA molecule; and (b) sequencing the TSP-hybridized RNA molecule or a derivative thereof.

The methods, compositions, and kits disclosed herein can be used to quantify an RNA molecule. Generally, the methods, compositions, and kits comprise: (a) hybridizing one or more TSPs to an RNA molecule to produce a TSP-hybridized RNA molecule; and (b) detecting the TSP-hybridized RNA molecule or a derivative thereof, thereby quantifying the RNA molecule.

In some instances, the methods, compositions, and kits disclosed herein can reduce or prevent the formation of a secondary structure in the RNA molecule. The methods, compositions, and kits generally comprise hybridizing one or more TSPs to a RNA molecule to produce a TSP-hybridized RNA molecule, thereby preventing the formation of a secondary structure in the RNA molecule.

As used herein, the terms “derivative of an RNA molecule”, “RNA molecule derivative”, “product of an RNA molecule” are used interchangeably and refer to any product or derivative of a RNA molecule disclosed herein. In some instances, derivatives of RNA molecule comprise the products of a reaction comprising a RNA molecule. For example, derivatives of RNA molecules include, but are not limited to, TSP-hybridized RNA molecule, cDNA, amplified RNA molecule, circularized RNA, released RNA molecule, etc.

### I. Target-Specific Oligonucleotide Probes (TSPs)

The methods, compositions, and systems disclosed herein may comprise one or more target-specific oligonucleotide probes (TSPs). As used herein, the terms “target-specific oligonucleotide probe” (“TSP”) or capture probe may be used interchangeably. A TSP is an oligonucleotide that can hybridize to a RNA molecule as disclosed herein. The TSPs disclosed herein can comprise one or more nucleotide residues selected from: deoxyribonucleic acid (DNA), ribonucleic acid (RNA), chemically modified sugar derivatives of DNA or RNA (e.g., 2′-OMe, or 2′-fluoro (2′-F), chemically modified nucleobase derivatives of DNA or RNA, abasic sites, a mimetic of DNA or RNA, and any combination thereof. In some instances, the TSPs further comprise one or more non-natural analogs. The non-natural analogs include, but are not limited to, peptide nucleic acid (PNA) linkages and Locked Nucleic Acid (LNA) linkages. In some instances, at least one TSP comprises a sequence selected from any of SEQ ID NOs: 49-60, or a portion thereof.

In some instances, the TSP comprises a RNA-specific portion or segment. In some instances, the TSP comprises only a RNA-specific portion or segment. The RNA-specific portion or segment of the TSP may be at least partially complementary to the RNA molecule. The length of RNA-specific portion can range from about 4 nucleotides up to the full length of the RNA molecule. The length of RNA-specific portion can range from about 8 nucleotides to the full length of the RNA molecule. The length of RNA-specific portion can range from about 10 nucleotides to the full length of the RNA molecule. The length of RNA-specific portion can range from about 12 nucleotides to the full length of the RNA molecule. The length of RNA-specific portion can range from about 14 nucleotides to the full length of the RNA molecule. The length of RNA-specific portion can range from about 16 nucleotides to the full length of the RNA molecule. In some instances, the length of the RNA-specific portion is between about 13 to about 22 nucleotides. In other instances, the length of the RNA-specific portion is between about 15 to about 22 nucleotides. The length of the RNA-specific portion can be between 17 to about 22 nucleotides. In some instances, the length of the RNA-specific portion is between about 13 to about 20 nucleotides. In other instances, the length of the RNA-specific portion is between about 15 to about 20 nucleotides. The length of the RNA-specific portion can be between 17 to about 20 nucleotides. In some instances, the length of the RNA-specific portion is between about 13 to about 19 nucleotides. In other instances, the length of the RNA-specific portion is between about 15 to about 19 nucleotides. The length of the RNA-specific portion can be between 17 to about 19 nucleotides. In some instances, the length of the RNA-specific portion is between about 13 to about 18 nucleotides. In other instances, the length of the RNA-specific portion is between about 15 to about 18 nucleotides. The length of the RNA-specific portion can be between 17 to about 18 nucleotides.

In some instances, the sequence of the RNA-specific portion of the TSP is at least partially complementary to at least a portion of the sequence of the RNA molecule. The sequence of the RNA-specific portion can be at least about 50% to about 100% complementary to at least a portion of the sequence of the RNA molecule. In some instances, the sequence of the RNA-specific portion is at least about 50% complementary to the sequence of the RNA molecule. In some instances, the sequence of the RNA-specific portion is at least about 60% complementary to the sequence of the RNA molecule. In some instances, the sequence of the RNA-specific portion is at least about 65% complementary to the sequence of the RNA molecule. In some instances, the sequence of the RNA-specific portion is at least about 70% complementary to the sequence of the RNA molecule. In some instances, the sequence of the RNA-specific portion is at least about 75% complementary to the sequence of the RNA molecule. Alternatively, the sequence of the RNA-specific portion is at least about 80% complementary to the sequence of the RNA molecule. In some instances, the sequence of the RNA-specific portion is at least about 85% complementary to the sequence of the RNA molecule. In some instances, the sequence of the RNA-specific portion is at least about 90% complementary to the sequence of the RNA molecule. Alternatively, the sequence of the RNA-specific portion is at least about 95% complementary to the sequence of the RNA molecule. The sequence of the RNA-specific portion can be at least about 97% complementary to the sequence of the RNA molecule. In some instances, the sequence of the RNA-specific portion is at least about 98% complementary to the sequence of the RNA molecule. In other instances, the sequence of the RNA-specific portion is at least about 99% complementary to the sequence of the RNA molecule.

The sequence of the RNA-specific portion can comprise about 5 or fewer mismatches from at least a portion of the sequence of the RNA molecule. The sequence of the RNA-specific portion can comprise about 4 or fewer mismatches from at least a portion of the sequence of the RNA molecule. The sequence of the RNA-specific portion can comprise about 3 or fewer mismatches from at least a portion of the sequence of the RNA molecule. The sequence of the RNA-specific portion can comprise about 2 or fewer mismatches from at least a portion of the sequence of the RNA molecule. The sequence of the RNA-specific portion can comprise about 1 or fewer mismatches from at least a portion of the sequence of the RNA molecule.

The TSP may further comprise a linker. The linker may be a single-stranded overhang at the 3′ and/or 5′ end of the TSP in the TSP-hybridized RNA molecule. The linker may comprise a sequence that is non-complementary to RNA molecule or primers. The linker may comprise one or more nucleotides, non-nucleotides, or a combination thereof. The linker may be used to distance the RNA-specific portion of the TSP from the surface of the solid support. The linker may enable hybridization or attachment of the TSP to the solid support. The linker can improve the efficiency of hybridization between TSP and RNA molecule. The linker may enable detection, quantification, and/or capture of the TSP or TSP-hybridized RNA molecule.

The linker may comprise a label. The label may be a ligand, small molecule, hapten, or anchor group or nanoparticle. The hapten may be biotin. The nanoparticle may be a gold nanoparticle. The ligand may be derivatives of polyhistidine, or EDTA, or o-phenanthroline.

In some instances, the length of TSP is at least about 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, or 200 or more nucleotides. In other instances, the length of the TSP can be at least about 14 nucleotides. In other instances, the length of the TSP is at least about 15 nucleotides. Alternatively, the length of the TSP is at least about 16 nucleotides. The length of the TSP can be at least about 17 nucleotides. In some instances, the length of the TSP is at least about 18 nucleotides. In other instances, the length of the nucleotide is at least about 19 nucleotides. Alternatively, the length of the TSP is at least about 20 nucleotides.

In some embodiments, at least one TSP is hybridized to the RNA molecule. In other embodiments, two or more TSPs are hybridized to different regions of the same RNA molecule. Alternatively, three or more TSPs are hybridized to different regions of the same RNA molecule.

In some instances, at least about two TSPs are hybridized to the RNA molecules. In other instances, at least about three TSPs are hybridized to the RNA molecules. Alternatively, or additionally, at least about five TSPs are hybridized to the RNA molecules. In some instances, at least about ten TSPs are hybridized to the RNA molecules. The TSPs can comprise the same sequence. Alternatively, at least two TSPs comprise different sequences. The TSPs can hybridize to copies of the same RNA molecule. Alternatively, the TSPs can hybridize to at least two different RNA molecules.

In some instances, the TSP hybridizes to the RNA molecule to produce a TSP-hybridized RNA molecule, wherein the TSP-hybridized RNA molecule comprises one or two single-stranded overhangs on the RNA molecule. In some instances, the overhangs comprise non-hybridized regions on the RNA molecule. In some instances, the TSP-hybridized RNA molecule comprises an overhang at only one end of the RNA molecule. In other instances, the TSP-hybridized RNA molecule comprises overhangs at both ends of the RNA molecule. The overhang can be at the 5′ end of the RNA molecule (5′-overhang). Alternatively, or additionally, the overhang is at the 3′ end of the RNA molecule (3′-overhang). The TSP-hybridized RNA molecule can comprise an overhang at the 5′ end of the RNA molecule and an overhang at the 3′ end of the RNA molecule.

The methods, compositions, and systems disclosed herein can comprise a plurality of TSPs. In some instances, the plurality of TSPs comprises identical TSPs. For example, the plurality of TSPs comprises TSPs comprising the same sequence and length.

In other instances, the plurality of TSPs comprises two or more different TSPs. For example, the two or more different TSPs can comprise different sequences. In another example, the two or more different TSPs can comprise different lengths. In some instances, the two or more different TSPs comprise different sequences and different lengths.

The plurality of TSPs can comprise at least about two or more different TSPs. In other instances, the plurality of TSPs comprise at least about three or more different TSPs. Alternatively, the plurality of TSPs comprise at least about four or more different TSPs. In some instances, the plurality of TSPs comprise at least about four or more different TSPs. The plurality of TSPs can comprise at least about five or more different TSPs. In other instances, the plurality of TSPs comprise at least about six or more different TSPs. Alternatively, the plurality of TSPs comprise at least about seven or more different TSPs. In some instances, the plurality of TSPs comprise at least about eight or more different TSPs. The plurality of TSPs can comprise at least about ten or more different TSPs. In other instances, the plurality of TSPs comprise at least about fifteen or more different TSPs. Alternatively, the plurality of TSPs comprise at least about twenty or more different TSPs. In some instances, the plurality of TSPs comprise at least about 25, 30, 35, 40, 45, 50, 55, 60, 65, 70, 75, 80, 85, 90, 95, 100 or more different TSPs. In other instances, the plurality of TSPs comprise at least about 150, 200, 250, 300, 350, 400, 450, 500, 550, 600, 650, 700, 750, 800, 850, 900, 950, 1000 or more different TSPs.

The TSPs for use in the methods, compositions, and kits disclosed herein can further comprise one or more blocking groups. In some instances, the TSP comprises a blocking group at its 5′ end (e.g., 5′-end blocking group). In other instances, the TSP comprises a blocking group at its 3′ end (e.g., 3′-end blocking group). Alternatively, the TSP comprises a blocking group at its 5′ end and its 3′ end.

The TSPs for use in the methods, compositions, and kits disclosed herein can further comprise one or more blocking groups. In some instances, the TSP comprises a blocking group at its 5′ end (e.g., 5′ blocking group). In other instances, the TSP comprises a blocking group at its 3′ end (e.g., 3′ blocking group). Alternatively, the TSP comprises a blocking group at its 5′ end and its 3′ end.

In some instances, the blocking group comprises a termination group that is a 3′-phosphate (3′-p or Np); a 3′-amino; a 2′,3′-dideoxy nucleoside (ddN); a 3′- inverted (3′-3′) deoxynucleoside (idN); a 3′-inverted abasic site; or a 3′-non-nucleoside linker (n-linker). In some embodiments, the TSP comprises a blocking group at its 5′ end that prevents its phosphorylation, e.g., a 5′-OMe or a non-nucleotide linker. In some embodiments, the TSP comprises one or more residues that cannot be replicated by DNA polymerase; e.g., an abasic site(s) or nucleoside(s) with 2′-OMe or 2′-F modifications.

In some instances, the 3′ blocking group on the TSP prevents extension of the 3′ end of TSP. In some instances, the 3′ blocking group on the TSP prevents extension of the 3′ end of TSP by a reverse transcriptase. In other instances, the 3′ blocking group on the TSP prevents extension of the 3′ end of TSP by a DNA polymerase.

### II. Nucleic Acid Molecules

The methods, compositions and systems discloses herein may comprise hybridization of one or more TSPs to one or more nucleic acid molecules to produce one or more TSP-hybridized nucleic acid molecules. The one or more nucleic acid molecules may comprise 200 or fewer nucleotides. The one or more nucleic acid molecules may comprise a fragment of a nucleic acid molecule. The one or more nucleic acid molecules may be an RNA and/or DNA-RNA hybrid, or derivatives thereof.

The methods, compositions and systems disclosed herein often comprise hybridization of a TSP to a RNA molecule. As used herein, a “RNA molecule” is a small RNA molecule. In some instances, a RNA molecule or a small RNA molecule can also be referred to as a non-coding RNA. A non-limiting list of RNA molecules includes microRNAs (miRNAs), siRNA, shRNA, piRNA, tasiRNA, rasiRNA, scnRNA, tiRNA, smRNA, tncRNA, ncRNA, snRNA, snoRNA, scnRNA, qiRNA, and pre-miRNAs. RNA molecules can also include fragments of larger coding RNAs (e.g., mRNA or viral RNAs) or non-coding RNAs (e.g., ribosomal RNAs, lncRNAs, pri-miRNAs). In some instances, the RNA molecule is a miRNA. In other instances, the RNA molecule is a siRNA or shRNA. The RNA molecule can be an ncRNA. The RNA molecule can be a small ncRNA.

Generally, a RNA molecule disclosed herein comprises an RNA molecule with one or more of the following features: (a) size ranging from 5 to 200 nucleotides; (b) 5′ ends selected from the group comprising 5′-phospate (5′-p); and 5′-hydroxyl (5′-OH), or 5′-cap, or 5′-triphosphate (5′-ppp); (c) 3′ groups at the 3′ ends selected from group comprising 3′-hydroxyl (3′-OH), 3′-phospate (3′-p), or 2′,3′-cyclic phosphate (2′,3′>p); and/or (d) 2′ groups at the 3′ ends selected from the group comprising 2′-OH or 2′-oxymethyl (2′-OMe). In some instances, the choice for 5′-p and 3′-OH ends, which can be naturally occurring in miRNAs, is based on substrate requirements for enzymatic ligation and extension reactions.

The RNA molecule may be equal to or less than about 200 nucleotides or base pairs. The RNA molecule may be equal to or less than about 200, 190, 180, 170, 160, 50, 140, 130, 120, 110, 100, 95, 90, 85, 80, 75, 70, 65, 60, 55, 50, 45, 40, 35, 30, 29, 28, 27, 26, 25 or few nucleotides or base pairs.

In some instances, the length of the RNA molecule is between about 10 to about 100 nucleotides. In other instances, the length of the RNA molecule is between about 15 to about 125 nucleotides. Alternatively, the length of the RNA molecule is between about 15 to about 100 nucleotides. The length of the RNA molecule can be between about 15 to about 90 nucleotides. In some instances, the length of the RNA molecule is between about 17 to about 80 nucleotides. In other instances, the length of the RNA molecule is between about 17 to about 70 nucleotides. Alternatively, the length of the RNA molecule is between about 17 to about 60 nucleotides. The length of the RNA molecule can be between about 17 to about 50 nucleotides. In some instances, the length of the RNA molecule is between about 19 to about 40 nucleotides. In other instances, the length of the RNA molecule is between about 19 to about 30 nucleotides. Alternatively, the length of the RNA molecule is between about 19 to about 25 nucleotides. The length of the RNA molecule can be between about 19 to about 23 nucleotides. In some instances, the length of the RNA molecule is between about 20 to about 25 nucleotides. In other instances, the length of the RNA molecule is between about 20 to about 24 nucleotides. Alternatively, the length of the RNA molecule is between about 20 to about 23 nucleotides. The length of the RNA molecule can be between about 20 to about 22 nucleotides. In some instances, the length of the RNA molecule is between about 21 to about 25 nucleotides. In other instances, the length of the RNA molecule is between about 21 to about 24 nucleotides. Alternatively, the length of the RNA molecule is between about 21 to about 23 nucleotides. The length of the RNA molecule can be between about 21 to about 22 nucleotides.

The length of the RNA molecule can be at least about 17 nucleotides. In some instances, the length of the RNA molecule is at least about 18 nucleotides. In other instances, the length of the RNA molecule is at least about 19 nucleotides. Alternatively, the length of the RNA molecule is at least about 20 nucleotides. The length of the RNA molecule can be at least about 21 nucleotides. In some instances, the length of the RNA molecule is at least about 22 nucleotides. In other instances, the length of the RNA molecule is at least about 23 nucleotides. Alternatively, the length of the RNA molecule is at least about 24 nucleotides. The length of the RNA molecule can be at least about 25 nucleotides. In some instances, the length of the RNA molecule is at least about 26 nucleotides. In other instances, the length of the RNA molecule is at least about 27 nucleotides. Alternatively, the length of the RNA molecule is at least about 28 nucleotides.

The length of the RNA molecule can be less than about 30 nucleotides. In some instances, the length of the RNA molecule is less than about 29 nucleotides. In other instances, the length of the RNA molecule is less than about 28 nucleotides. Alternatively, the length of the RNA molecule is less than about 27 nucleotides. The length of the RNA molecule can be less than about 26 nucleotides. In some instances, the length of the RNA molecule is less than about 25 nucleotides. In other instances, the length of the RNA molecule is less than about 24 nucleotides. Alternatively, the length of the RNA molecule is less than about 23 nucleotides. The length of the RNA molecule can be less than about 22 nucleotides. In some instances, the length of the RNA molecule is less than about 21 nucleotides. In other instances, the length of the RNA molecule is less than about 20 nucleotides. Alternatively, the length of the RNA molecule is less than about 19 nucleotides. The length of the RNA molecule can be less than about 18 nucleotides. In some instances, the length of the RNA molecule is less than about 17 nucleotides. In other instances, the length of the RNA molecule is less than about 16 nucleotides. Alternatively, the length of the RNA molecule is less than about 15 nucleotides.

The methods, compositions, and kits disclosed herein can comprise hybridizing a plurality of TSPs to a plurality of RNA molecules. In some instances, the plurality of RNA molecules comprises RNA molecules of identical sequences. In other instances, the plurality of RNA molecules comprises different RNA molecules. The different RNA molecules can comprise different sequences. Alternatively, the different RNA molecules comprise different lengths. In other instances, the different RNA molecules comprise different isoforms of a RNA molecule. The different RNA molecules can comprise different isomirs of a RNA molecule. The plurality of RNA molecules can comprise the same type of small RNA molecule. For example, the plurality of RNA molecules can comprise miRNAs. In another example, the plurality of RNAs can comprise siRNAs. Alternatively, the plurality of RNAs can comprise different types of small RNA molecules. For example, the plurality of RNA molecules can comprise miRNAs and siRNAs.

The one or more RNA molecules may be low-copy RNA molecules or low abundance RNA molecules. The low-copy RNA molecules or low abundance RNA molecules may comprise 30%, 25%, 20%, 15%, 14%, 13%, 12%, 11%, 10%, 9%, 8%, 7%, 6%, 5%, 4%, 3%, 2%, 1% or less than the total number of nucleic acid molecules in the sample. The low-copy RNA molecules or low abundance RNA molecules may comprise 15% or less than the total number of nucleic acid molecules in the sample. The low-copy RNA molecules or low abundance RNA molecules may comprise 10% or less than the total number of nucleic acid molecules in the sample. The low-copy RNA molecules or low abundance RNA molecules may comprise 5% or less than the total number of nucleic acid molecules in the sample. The low-copy RNA molecules or low abundance RNA molecules may comprise 2% or less than the total number of nucleic acid molecules in the sample. The low-copy RNA molecules or low abundance RNA molecules may comprise 1% or less than the total number of nucleic acid molecules in the sample. The low-copy RNA molecules or low abundance RNA molecules may comprise 0.5% or less than the total number of nucleic acid molecules in the sample. The low-copy RNA molecules or low abundance RNA molecules may comprise 0.2% or less than the total number of nucleic acid molecules in the sample.

The low-copy RNA molecules or low abundance RNA molecules may comprise 50%, 40%, 35%, 30%, 25%, 20%, 15%, 14%, 13%, 12%, 11%, 10%, 9%, 8%, 7%, 6%, 5%, 4%, 3%, 2%, 1% or less than the total number of RNA molecules in the sample. The low-copy RNA molecules or low abundance RNA molecules may comprise 20% or less than the total number of RNA molecules in the sample. The low-copy RNA molecules or low abundance RNA molecules may comprise 15% or less than the total number of RNA molecules in the sample. The low-copy RNA molecules or low abundance RNA molecules may comprise 10% or less than the total number of RNA molecules in the sample. The low-copy RNA molecules or low abundance RNA molecules may comprise 5% or less than the total number of RNA molecules in the sample. The low-copy RNA molecules or low abundance RNA molecules may comprise 2% or less than the total number of RNA molecules in the sample. The low-copy RNA molecules or low abundance RNA molecules may comprise 1% or less than the total number of RNA molecules in the sample. The low-copy RNA molecules or low abundance RNA molecules may comprise 0.5% or less than the total number of RNA molecules in the sample. The low-copy RNA molecules or low abundance RNA molecules may comprise 0.2% or less than the total number of RNA molecules in the sample.

The low-copy RNA molecules or low abundance RNA molecules may have 1000, 900, 800, 700, 600, 500, 400, 300, 200, 100 or fewer copies in the sample. The low-copy RNA molecules or low abundance RNA molecules may have 500or fewer copies in the sample. The low-copy RNA molecules or low abundance RNA molecules may have 450 or fewer copies in the sample. The low-copy RNA molecules or low abundance RNA molecules may have 400 or fewer copies in the sample. The low-copy RNA molecules or low abundance RNA molecules may have 350 or fewer copies in the sample. The low-copy RNA molecules or low abundance RNA molecules may have 300or fewer copies in the sample. The low-copy RNA molecules or low abundance RNA molecules may have 250 or fewer copies in the sample. The low-copy RNA molecules or low abundance RNA molecules may have 200 or fewer copies in the sample. The low-copy RNA molecules or low abundance RNA molecules may have 150 or fewer copies in the sample. The low-copy RNA molecules or low abundance RNA molecules may have 100 or fewer copies in the sample. The low-copy RNA molecules or low abundance RNA molecules may have 90 or fewer copies in the sample. The low-copy RNA molecules or low abundance RNA molecules may have 80 or fewer copies in the sample. The low-copy RNA molecules or low abundance RNA molecules may have 70 or fewer copies in the sample. The low-copy RNA molecules or low abundance RNA molecules may have 60 or fewer copies in the sample. The low-copy RNA molecules or low abundance RNA molecules may have 50 or fewer copies in the sample. The low-copy RNA molecules or low abundance RNA molecules may have 40 or fewer copies in the sample. The low-copy RNA molecules or low abundance RNA molecules may have 30 or fewer copies in the sample. The low-copy RNA molecules or low abundance RNA molecules may have 20 or fewer copies in the sample. The low-copy RNA molecules or low abundance RNA molecules may have 10 or fewer copies in the sample.

In some instances, the RNA molecules are low-copy RNA molecules. In some instances, the methods, compositions, and kits facilitate detection of low-copy RNA molecules. In some instances, the total number of low-copy RNA molecules detected in a reaction comprising one or more TSPs is at least about 10%, 20%, 25%, 30%, 35%, 40%, 45%, 45%, or 50% higher than the total number of low-copy RNA molecules detected in a reaction that does not comprise one or more TSPs. In other instances, the total number of low-copy RNA molecules detected in a reaction comprising one or more TSPs is at least about 55% higher than the total number of low-copy RNA molecules detected in a reaction that does not comprise one or more TSPs. Alternatively, the total number of low-copy RNA molecules detected in a reaction comprising one or more TSPs is at least about 60% higher than the total number of low-copy RNA molecules detected in a reaction that does not comprise one or more TSPs. The total number of low-copy RNA molecules detected in a reaction comprising one or more TSPs can be at least about 65% higher than the total number of low-copy RNA molecules detected in a reaction that does not comprise one or more TSPs. In some instances, the total number of low-copy RNA molecules detected in a reaction comprising one or more TSPs is at least about 70% higher than the total number of low-copy RNA molecules detected in a reaction that does not comprise one or more TSPs. In other instances, the total number of low-copy RNA molecules detected in a reaction comprising one or more TSPs is at least about 75% higher than the total number of low-copy RNA molecules detected in a reaction that does not comprise one or more TSPs. Alternatively, the total number of low-copy RNA molecules detected in a reaction comprising one or more TSPs is at least about 80% higher than the total number of low-copy RNA molecules detected in a reaction that does not comprise one or more TSPs. The total number of low-copy RNA molecules detected in a reaction comprising one or more TSPs can be at least about 85% higher than the total number of low-copy RNA molecules detected in a reaction that does not comprise one or more TSPs. In some instances, the total number of low-copy RNA molecules detected in a reaction comprising one or more TSPs is at least about 90% higher than the total number of low-copy RNA molecules detected in a reaction that does not comprise one or more TSPs. In other instances, the total number of low-copy RNA molecules detected in a reaction comprising one or more TSPs is at least about 95% higher than the total number of low-copy RNA molecules detected in a reaction that does not comprise one or more TSPs. Alternatively, the total number of low-copy RNA molecules detected in a reaction comprising one or more TSPs is at least about 97% higher than the total number of low-copy RNA molecules detected in a reaction that does not comprise one or more TSPs. The total number of low-copy RNA molecules detected in a reaction comprising one or more TSPs can be at least about 99% higher than the total number of low-copy RNA molecules detected in a reaction that does not comprise one or more TSPs.

### III. Linkers or Spacers

In some embodiments, TSPs further comprise one or more linker or spacer segments. The linker or spacer segment comprises either nucleotides or non-nucleotide moieties, or combination of both. The linker may be used to distance the RNA-specific portion of the TSP from the surface of the solid phase. The linker may improve the efficiency of hybridization between the TSP and the RNA molecule. The linker may be used to attach the TSP to one or more solid supports.

The linker is not complementary to the RNA molecule or the RNA-specific RT or PCR primers, wherein the RNA-specific primers are complementary to at least a portion of the RNA-molecule hybridized to the TSP. The linker can comprise between about 1 to about 60 nucleotides. In some instances, the linker can comprise between about 1 to about 50 nucleotides. In some instances, the linker can comprise between about 1 to about 40 nucleotides. In some instances, the linker can comprise between about 1 to about 30 nucleotides. In other instances, the linker can comprise between about 1 to about 20 nucleotides. Alternatively, the linker can comprise between about 1 to about 10 nucleotides. In some instances, the overhang can comprise between about 1 to about 5 nucleotides. In other instances, the linker can comprise between about 1 to about 4 nucleotides. Alternatively, the linker can comprise between about 1 to about 3 nucleotides. The linker can also comprise between about 1 to about 2 nucleotides.

The linker or spacer can comprise non-nucleotide polymeric units that match the length of from 1 to 40 nucleotides. Examples of non-nucleotide linkers and spacers include ethylene glycol, peptide, ethyleneimine and others reviewed by Beaucage (2001).

IV. Amplification of RNA Molecules

The methods, compositions and kits as disclosed herein can further comprise amplification of a RNA molecule, or a RNA molecule-specific sequence corresponding to or complementary to a RNA molecule, or derivatives thereof, to produce an amplicons (e.g., amplified cDNA or amplified cRNA). In some instances, a RNA molecule-specific sequence comprises a sequence that is identical or complementary to at least to part of a RNA molecule sequence. In some instances, the amplicons are used for cloning into conventional sequencing vectors or for direct analysis by next-generation sequencing methods. In other instances, the amplicons are used for further amplification and detection of the RNA molecule-specific sequences by PCR-based methods. In some other instances, the amplicons are used for detection of the amplified sequences by other methods such as isothermal amplification methods.

Amplification of the RNA molecule or derivative thereof can comprise PCR-based methods. Examples of PCR-based methods include, but are not limited to, RT-PCR, end-point PCR, real-time qPCR, HD-PCR, digital PCR, or any combination thereof. Additional PCR methods include, but are not limited to, droplet PCR, emulsion PCR, overlap extension PCR (OE-PCR), inverse PCR, linear-after-the-exponential (LATE)-PCR, and MegaPlex PCR.

Alternatively, amplification of the RNA molecule or derivative thereof comprises isothermal amplification methods. Examples of these methods include, but are not limited to, multiple displacement amplification (MDA), transcription-mediated amplification (TMA), nucleic acid sequence-based amplification (NASBA), strand displacement amplification (SDA), real-time SDA, rolling circle amplification (RCA), hyperbranched RCA (HRCA) or circle-to-circle amplification.

In some instances, amplification of the RNA molecule or derivative thereof comprises (a) circularizing the RNA molecule or derivative thereof to produce a circularized RNA molecule; and (b) conducting a reaction to amplify the circularized RNA molecule-specific sequences. Conducting a reaction to amplify the circularized RNA molecule-specific sequences can comprise any amplification method. For example, conducting a reaction to amplify the circularized RNA molecule-specific sequences can comprise any of the amplification methods disclosed herein.

In other instances, amplification of the RNA molecule or derivative thereof comprises (a) conducting a reaction to reverse transcribe the RNA molecule or derivative thereof to produce a cDNA, wherein the cDNA is a DNA copy of the RNA molecule or derivative thereof; and (b) conducting a reaction to amplify the cDNA.

### V. Isolation of and/or Enrichment for RNA Molecules

The methods, compositions, and kits disclosed herein can further comprise isolation of and/or enrichment for a RNA molecule. The RNA molecule derivatives can be any of the forms or products of a RNA molecule as disclosed herein. In some instances, a RNA molecule derivative comprises a TSP-hybridized RNA molecule, RNA molecule, amplified RNA molecule, RNA amplicon, RNA molecule or any combination thereof.

In some instances, isolation and/or purification of a RNA molecule or derivative thereof comprises attachment of the RNA molecule or derivative thereof to one or more substrates. Attachment of the RNA molecule or derivative thereof can comprise immobilization of the RNA molecule or derivative thereof to the one or more substrates. As used herein, the term “substrate” refers to a material or group of materials having a rigid or semi-rigid surface or surfaces. The substrate can be a solid support or phase. Alternatively, the substrate is a non-solid support. In some instances, the support comprises a membrane, paper, plastic, coated surface, flat surface, glass, slide, chip, or any combination thereof. In some instances, at least one surface of the solid support will be substantially flat. Alternatively, the substrate comprises physically separate synthesis regions for different compounds with, for example, wells, raised regions, pins, etched trenches, or the like. In other instances, the substrate comprises beads, resins, gels, microspheres, or other geometric configurations. Alternatively, the substrates comprise silica chips, microparticles, nanoparticles, plates, and arrays. In some instances, isolation and/or purification of the RNA molecule or derivative thereof comprises hybridization of the RNA molecule or derivative thereof to the substrate. In some instances, isolation and/or purification of the RNA molecule or derivative thereof comprises the use of one or more beads. In some instances, the beads are magnetic and/or streptavidin-coated beads. In some instances, the beads are beads coated by antibodies specific to a hapten group attached to the TSP.

In some instances, isolation and/or purification of the RNA molecule or derivative thereof comprises one or more wash steps. For example, isolation and/or purification can comprise (a) attaching one or more TSP-hybridized RNA molecules or derivative thereof to a substrate; (b) applying a wash buffer to the substrate; and (c) removing the wash buffer and unbound molecules, thereby isolating and/or purifying the RNA molecule or derivative thereof. Isolation and/or purification of the small RNA molecule or derivative thereof may comprise positive selection. For example, the small RNA molecules of interest are hybridized to a plurality of TSPs to produce TSP-hybridized RNA molecules. Positive selection may comprise capturing the TSP-hybridized RNA molecules onto a substrate and removing non-hybridized molecules.

### VI. Detection and/or Quantification of RNA Molecules

The methods, compositions, and systems disclosed herein can further comprise detection and/or quantification of a RNA molecule or derivative thereof. In some instances, the derivative of the RNA molecule comprises a TSP-hybridized RNA molecule, RNA molecule, amplified RNA molecule, amplicon, cDNA, circularized RNA molecule, linear RNA molecule, released RNA molecule, free RNA molecule, or any combination thereof. In some instances, the number of the derivative of the RNA molecules detected directly corresponds to the number of RNA molecules.

In some instances, detection and/or quantification of the RNA molecule or derivative thereof comprises conducting one or more RT-qPCR assays. In some instances, conducting one or more RT-qPCR assays comprises performing a TaqMan microRNA assay (Applied Biosystems/Life Technologies), mirVana RT-qPCR assay (Ambion/Life Technologies), miR-ID assay (Somagenics), miRNA 3′-end polyadenylation-based assays such as miScript (SABiosciences/Qiagen), miRCURY (Exiqon) or other assays known in art RT-qPCR assays adapted for small RNA detection.

In some instances, detection and/or quantification of the RNA molecule or derivative thereof comprises conducting hybridization-based including but not limited to p19 miRNA Detection (NEB), NCounter (Nanostring), and nanopore single-molecule detection (Gu et al. 2012)

In some instances, detection and/or quantification of the RNA molecule or derivative thereof comprises conducting a sequencing reaction to determine the sequence of at least a portion of the RNA molecule or derivative thereof. Conducting a sequencing reaction can comprise next- (or second-, or third-) generation sequencing (NGS) technologies. In other instances, conducting a sequencing reaction can comprise third-generation sequencing such as direct, single-molecule sequencing. In some instances, conducting a sequencing reaction comprises Solexa sequencing (Illumina), 454 pyrosequencing (Roche), SOLiD sequencing and Ion Torrent (both from Life Technologies), Nanopore DNA sequencing, Lynx Therapeutics' Massively Parallel Signature Sequencing (MPSS), Single Molecule real time (RNAP) sequencing, Ion semiconductor sequencing, Single Molecule SMRT sequencing, Polony sequencing, DNA nanoball sequencing, and real-time single molecule sequencing. Alternatively, conducting a sequencing reaction uses one or more sequencing instruments, including, but not limited to, Genome Analyzer IIx, HiSeq, and MiSeq offered by Illumina, Single Molecule Real Time (SMRT) technology, such as the PacBio RS system (Pacific Biosciences) and the Solexa Sequencer, and True Single Molecule Sequencing (tSMS) technology such as the HeliScope single molecule sequencing (Helicos).

Conducting a sequencing reaction can comprise paired-end sequencing, nanopore sequencing, high-throughput sequencing, shotgun sequencing, dye-terminator sequencing, multiple-primer DNA sequencing, primer walking, Sanger dideoxy sequencing, Maxim-Gilbert sequencing, pyrosequencing, true single molecule sequencing, or any combination thereof. Alternatively, the sequence of the labeled molecule or any product thereof can be determined by electron microscopy or a chemical-sensitive field effect transistor (chemFET) array.

### VII. Exemplary Embodiments

A detailed description regarding various aspects of the invention is provided herein using miRNAs as examples. However, these embodiments can be applied equally well to other small RNAs such as miRNAs, siRNA, shRNA, piRNA, tasiRNA, rasiRNA, scnRNA, tiRNA, smRNA, tncRNA, ncRNA, snRNA, snoRNA, scnRNA, qiRNA, or small fragments of larger RNAs. The term “RNA molecules” may refer to any of these small RNAs.

Methods for analyzing one or more RNA molecules can comprise contacting a sample comprising a plurality of molecules with one or more target-specific oligonucleotide probes (TSPs) to produce one or more TSP-hybridized RNA molecules, wherein (i) the TSP comprises a RNA-specific portion that is at least partially complementary to one or more RNA molecules; and (ii) a TSP-hybridized RNA molecule is produced from hybridization of the TSP to the RNA molecule.

Further provided herein are methods for expression profiling of one or more RNA molecules comprising (a) hybridizing one or more TSPs to one or more RNA molecules to produce one or more TSP-hybridized RNA molecules; and (b) capturing the one or more TSP-hybridized RNA molecules by attaching the one or more TSP-hybridized RNA molecules to a solid phase/support to produce one or more captured TSP-hybridized RNA molecules; (c) purifying or isolating the one or more captured TSP-hybridized RNA molecules; (d) releasing the one or more captured TSP-hybridized RNA molecules into solution to produce one or more released RNA molecules, wherein the one or more released RNA molecules are circularized; and (e) detecting the one or more released RNA molecules in solution. Purifying may comprise removing the supernatant from the sample comprising the captured TSP-hybridized RNA molecules. Purifying the one or more captured TSP-hybridized RNA molecules may comprise washing the one or more captured TSP-hybridized RNA molecules on a solid phase or solid support under conditions providing stability of the one or more TSP-hybridized RNA molecules. Releasing the captured TSP-hybridized RNA molecules may comprise dissociating the RNA molecule from the TSP-hybridized RNA molecules. The released RNA molecules may comprise RNA molecules that are not hybridized to the TSPs. Alternatively, the released RNA molecules comprise RNA molecules that are hybridized to the TSPs. Detecting the one or more released RNA molecules may comprise quantifying the one or more RNA molecules.

In some instances, the methods further comprise conducting a sequence reaction on the RNA molecule or derivative thereof. The methods, compositions, and systems disclosed herein can reduce the amount of irrelevant sequencing reads. The methods, compositions, and systems can reduce the amount of irrelevant sequencing reads by at least about 50%. In some instances, the methods, compositions, and systems disclosed herein improve analysis of the samples. In some instances, the methods, compositions, and systems facilitate detection of low-copy RNA molecules.

The TSPs may be designed to avoid possible interference with one or more reactions (e.g., ligation, circularization, reverse transcription, PCR amplification, transcription, RCA or other kinds of isothermal amplification, etc). In some instances, the TSPs are designed in the ways that they cannot (a) serve as template for extension of the 3′-end of the RNA molecule; (b) serve as a splint in ligation of RNA molecules to adapters; (c) serve as a primer; (d) be ligated or extended; and/or (e) serve as a template for amplification. For example, the TSP is designed to not produce a single-stranded overhang at the 5′ end of the TSP upon hybridization to the RNA molecule. The TSPs may be designed to not produce a single-stranded overhang at the 5′ end of the TSP upon hybridization to the RNA molecule. The TSP may be designed to avoid accidental complementarity of the TSP to the linkers. The TSP may be designed to contain one or more blocking groups. The one or more blocking groups may be at the 3′ end of the TSP, 5′ end of the TSP, or a combination thereof. The blocking group at the 3′ end of the TSP may prevent the TSP from serving as a primer. The blocking group at the 5′ end may prevent 5′ phosphorylation. The blocking group may prevent one or more modifications to the TSP. The TSP may be designed to avoid complementarity to the primers (e.g., RT primers, amplification primers). The TSP may be designed to contain one or more residues that cannot be replicated by DNA polymerases. The TSP may be designed to comprise one or more abasic site(s) or nucleoside(s) with 2′-OMe or 2′-F modifications. The TSP may be designed to comprise an internal, stable hairpin.

In some instances, TSPs comprise one or more of the following features (a) the RNA-specific portion of the TSP is at least partially complementary to at least a portion of a RNA molecule; (b) the TSP can bind to more than one isoform of an RNA molecule; (c) the TSP can bind to more than one isomer of an RNA molecule; (d) the TSP comprises one or more nucleotides selected from: RNA; DNA; a mix of DNA and RNA residues, or modified nucleotides such as 2′-OMe, or 2′-fluoro (2′-F), locked nucleic acid (LNA), abasic sites or any other nucleic acid modifications known in the art; and/or (e) the TSP has blocked 3′-ends such as 3′-p, or 3′-amino, or 2′, 3′-dideoxy nucleoside (ddN), 3′-inverted (3′-3′) deoxy nucleoside (idN), or any other modification known in the art that prevents ligation to or extension of the 3′ end. The number of complementary base pairs (bp) between the RNA-specific portion of the TSP and RNA molecule may be equal to or less than the full length of the RNA molecule. The TSP-hybridized RNA molecule may comprise one or more mismatches. Hybridization of the TSP to the RNA molecule may not require perfect sequence-specificity.

The specific designs of the TSP may vary depending on the RNA molecule sequence, conditions of circularization, reverse transcription, or other reactions.

As shown in FIG. 1, a plurality of TSPs targeting one or more known and/or predicted RNA molecules can be added to a sample to produce one or more TSP-hybridized RNA molecules (105). The sample may be an extract from one or more tissues, cells, lysates from tissues, lysates from cells, fluids, extracellular fluids, cell extracts, tissue extracts, RNA extracts, nucleic acid extracts, purified extracts, or a combination thereof. The sample may comprise one or more synthetic nucleic acid molecules (e.g., synthetic miRNA).

In some instances, the “solid-phase capture” function of TSP is exploited. TSPs may be attached to one or more solid supports. Attachment may comprise hybridization of the TSPs to one or more solid supports. Attachment of the TSPs may occur prior to hybridization of the TSP to the one or more RNA molecules. The modified TSPs can be immobilized on a solid support and used for affinity capture of RNA molecules. The RNA molecules may be from total RNA extracts, directly from lysates (cell or tissue) or from bodily fluids. Examples of solid supports include: beads (either non-magnetic or magnetic), membranes, filters, slides, microtiter plates, or microcapillaries made from various materials such as glass/silica, plastic, nitrocellulose, nylon, gold or other metal compounds.

In certain embodiments of this invention, TSPs are immobilized through non-covalent attachment of the modified TSP to a solid support. Examples of modifications to the TSP include, but are not limited to, (a) a hapten group such as biotin or digoxigenin that is attached to one of the TSP ends or internally, via non-nucleotide spacers or oligonucleotide linkers, and which can bind with high affinity to a surface-bound hapten-specific protein such as streptavidin or a hapten-specific antibody; (b) a 5′- or 3′-end oligonucleotide linker that is complementary to a capture oligonucleotide probe (COP) immobilized on a solid support. In other embodiments of this invention, the modified TSP is immobilized through covalent attachment to an appropriately activated solid-phase material. Examples of TSP modifications include, but are not limited to, attachment of one or more anchor groups (such as phosphate, amino or thio) to the ends of non-nucleotide or oligonucleotide linkers that are attached to terminal or internal nucleotides of TSP.

In some embodiments, TSPs are hybridized with RNA molecules in solution (“solution hybridization”) followed by immobilization on a solid support.

In some embodiments, miRNAs are directly hybridized with immobilized TSP (“solid-phase hybridization”) and washed before subsequent enzymatic steps. This approach may allow for “solid-phase” capture of miRNAs directly from cell or tissue lysates, or from human bodily fluids (such as plasma, serum, saliva, urine). Washing of the captured miRNAs may allow their enrichment, concentration, and purification before the next enzymatic steps. The purification step may eliminate possible inhibitors of the circularization and/or RT-PCR reactions as well as non-RNA molecules. As shown in Steps 3 and 4a of FIG. 1, capture of TSP-hybridized RNA molecule on solid support can be followed by dissociation of the TSP-hybridized RNA molecules under denaturing conditions and release of the RNA molecule into solution phase. Release of the RNA molecules may result in circularization of the RNA molecules. The solution phase may then separated from the solid phase containing immobilized TSPs, and transferred to another reaction chamber (such as tube, or microtiter well, or microcapillary) to be used in amplification and quantification of the released RNA (e.g., circularized RNA).

As shown in Steps 3 and 4b of FIG. 1, capture of TSP-hybridized RNA molecule on solid support can be followed by circularization the RNA molecules with or without their simultaneous release into solution phase. Circularization of the RNA molecules may comprise one or more RNA ligases.

In some embodiments, the captured and/or released RNA molecules are reverse transcribed. Reverse transcription may comprise annealing one or more RT primers to the RNA molecule. The RT primers may be less than about 13, 12, 11, 10, 9, 8, 7, 6, 5, or 4 nucleotides in length. The RT primers may be less than about 10 nucleotides in length. The RT primers may be less than about 6 nucleotides in length. The RT primers may be between about 4 to about 10 nucleotides in length. The RT primers may be between about 4 to about 8 nucleotides in length. The RT primers may be at least partially complementary to the RNA molecules.

The RT primers may be extended by an RNA-dependent DNA polymerase (reverse transcriptase) or a DNA-dependent DNA polymerase (DNA polymerase) that can accept RNA as templates.

The methods, systems and kits may comprise the one or more DNA polymerases. The DNA polymerases may have one or more features selected from (a) strand-displacement (helicase) activity; and/or (b) high thermostability. Examples of DNA polymerases include, but are not limited to, M-MuLV and its mutated versions such SuperScript II and SuperScript III thermostable reverse transcriptases; rTth and Hot Multi-Taq thermostable DNA polymerases; and Klenow Fragment of DNA polymerase I (KF).

In some embodiments, detecting the TSP-hybridized RNA molecule comprises reverse transcribing the TSP-hybridized RNA molecule. Detecting the TSP-hybridized RNA molecule may be amplification-free. For example, detecting the TSP-hybridized RNA molecule comprises direct single-molecule RNA sequencing without amplification. Alternatively, detecting the TSP-hybridized RNA molecule may comprise amplification. For example, reverse transcription is followed by amplification of the cDNA molecule. Amplification may be PCR based or non-PCR based. Amplification may comprise reverse transcription-rolling circle amplification (RT-RCA).

In some embodiments, the reverse transcription by DNA polymerase with strand-displacement or 5′-exonuclease activity displaces the TSP and releases the cDNA (products of the primer extension) into solution, whereupon they are amplified by PCR. In other embodiments of this invention, the ligation or extension products bound to the immobilized TSP are released into solution (e.g., by washing with hot H₂O or a low-salt buffer) and the solution phase is separated from the solid phase before RT or RT-PCR step performed in solution.

In some instances, the sequence of the RNA-specific portion of the TSP is at least about 70% complementary to the sequence of the RNA molecule. In other instances, the sequence of the RNA-specific portion of the TSP is at least about 80% complementary to the sequence of the RNA molecule. Alternatively, the sequence of the RNA-specific portion of the TSP is at least about 85% complementary to the sequence of the RNA molecule. The sequence of the RNA-specific portion of the TSP can be at least about 90% complementary to the sequence of the RNA molecule. In some instances, the sequence of the RNA-specific portion of the TSP is at least about 95% complementary to the sequence of the RNA molecule. In other instances, the sequence of the RNA-specific portion of the TSP is at least about 97% complementary to the sequence of the RNA molecule.

The TSP can comprise RNA, DNA, modified analogs thereof, or combinations thereof. In some instances, the TSP comprises at least about 1 nucleotide that cannot be replicated by a DNA polymerase. In other instances, the TSP comprises at least about 1 nucleotide that cannot be reverse transcribed by a reverse transcriptase. The TSP can comprise one or more hairpins. In some instances, the hairpins cannot be bypassed by a polymerase. In some instances, the polymerase is a reverse transcriptase.

Further disclosed herein is a kit comprising: (a) one or more TSPs; (b) instructions for hybridizing the one or more TSPs to one or more RNA molecules, wherein the RNA molecule is equal or less than about 200 nucleotides or base pairs in length. The kit may further comprise one or more primers. The primers may hybridize to one or more RNA molecules. The primers may hybridize to a DNA copy of the one or more RNA molecules. The primers may be less than about 13, 12, 11, 10, 9, 8, 7, 6, 5, or 4 nucleotides in length. The primers may be less than about 10 nucleotides in length. The RT primers may be less than about 6 nucleotides in length. The primers may be between about 4 to about 13 nucleotides in length. The primers may be between about 9 to about 12 nucleotides in length. The primers may be at least partially complementary to the RNA molecules. The RNA molecule may be less than about 200 nucleotides in length. The RNA molecule may be less than about 100 nucleotides in length. In some instances, the RNA molecule comprises about 15 nucleotides to about 25 nucleotides. In other instances, the RNA molecule comprises about 17 nucleotides to about 23 nucleotides. The instructions for hybridizing the TSP to the RNA molecule can comprise instructions for reverse transcribing and/or amplifying the RNA molecule. In some instances, reverse transcribing and/or amplifying the RNA molecule comprises one or more primers. In other instances, the sequence of the TSP is not complementary to the sequence of the one or more primers. In some instances, the TSP cannot hybridize to the one or more primers.

Further disclosed herein is a method for isolating one or more nucleic acid molecules, comprising (a) contacting one or more samples comprising one or more nucleic acid molecules with one or more target-specific oligonucleotide probes (TSPs) to produce one or more TSP-hybridized nucleic acid molecules, wherein (i) the one or more TSPs hybridize to one or more nucleic acid molecules that may be 200 or fewer nucleotides or base pairs in length; (ii) the one or more TSPs may comprise a nucleic acid-specific portion that hybridizes to at least a portion of the one or more nucleic acid molecules; and (iii) the length of the nucleic acid-specific portion may be less than or equal to the full length of the nucleic acid molecule; and (b) selecting for the one or more TSP-hybridized nucleic acid molecules or a derivative thereof, thereby isolating the one or more nucleic acid molecules. Selecting for the one or more TSP-hybridized RNA molecules may comprise positively selecting for the one or more TSP-hybridized nucleic acid molecules by capturing the one or more TSP-hybridized nucleic acid molecules. Capturing the one or more TSP-hybridized nucleic acid molecules may comprise capturing the one or more TSP-hybridized nucleic acid molecules on one or more solid supports to produce one or more captured TSP-hybridized nucleic acid molecules. Capturing the one or more TSP-hybridized nucleic acid molecules may occur prior to detecting the one or more TSP-hybridized nucleic acid molecules. Positively selecting for the one or more TSP-hybridized nucleic acid molecules may comprise removing one or more analytes from the sample, wherein the one or more analytes may be not hybridized to the one or more TSPs. Removing the one or more analytes from the sample may comprise washing the one or more captured TSP-hybridized nucleic acid molecules. The method may further comprise dissociating one or more nucleic acid molecules from the one or more TSP-hybridized nucleic acid molecules to produce one or more non-hybridized nucleic acid molecules, wherein the non-hybridized nucleic acid molecules may be nucleic acid molecules that may be not longer hybridized to the one or more TSPs. The one or more nucleic acid molecules may be dissociated from the one or more TSP-hybridized molecules in a solution. The one or more non-hybridized nucleic acid molecules may be fluidically separated from the one or more TSPs. The method may further comprise circularizing the one or more TSP-hybridized nucleic acid molecules or derivative thereof to produce one or more circularized nucleic acid molecules. The one or more TSP-hybridized molecules may be circularized in a multiplex reaction. The one or more TSP-hybridized molecules may be circularized simultaneously. One or more TSP-hybridized nucleic acid molecules may be circularized sequentially. Two or more TSP-hybridized nucleic acid molecules or derivatives thereof may be circularized. Five or more TSP-hybridized nucleic acid molecules or derivatives thereof may be circularized. Ten or more TSP-hybridized nucleic acid molecules or derivatives thereof may be circularized. Fifteen or more TSP-hybridized nucleic acid molecules or derivatives thereof may be circularized. Twenty or more TSP-hybridized nucleic acid molecules or derivatives thereof may be circularized. Thirty or more TSP-hybridized nucleic acid molecules or derivatives thereof may be circularized. Forty or more TSP-hybridized nucleic acid molecules or derivatives thereof may be circularized. Circularizing may comprise one or more thermostable ligases. The thermostable ligase may be a RNA ligase. The thermostable ligase may be a DNA ligase. The thermostable ligase may be a DNA/RNA ligase that works on the both type of nucleic acids. The thermostable ligase may be a CircLigase or CircLigase II. The ends of the one or more TSP-hybridized molecules may overlap. In some instances, the ends of the one or more TSP-hybridized molecules do not overlap. Circularizing may comprise heating the sample to a temperature greater than the melting temperature (Tm) of the one or more TSPs. The temperature may be greater than or equal to 50° C. The temperature may be greater than or equal to 55° C. The temperature may be greater than or equal to 60° C. The temperature may be greater than or equal to 65° C. The temperature may be greater than or equal to 67° C. The temperature may be greater than or equal to 70° C. The temperature may be greater than or equal to 72° C. The temperature may be greater than or equal to 75° C. The temperature may be greater than or equal to 77° C. The temperature may be greater than or equal to 80° C. The one or more samples may be from one or more lysates, fluids, extracellular fluids, nucleic acid extracts, nucleic acid extracts, purified nucleic acid samples, purified nucleic acid samples, subsets of one or more nucleic acid samples, or a combination thereof. The one or more fluids may comprise secretions, sweat, tears, saliva, spinal fluid, blood, plasma, serum, ocular fluid, urine, or a combination thereof. The one or more samples may be from one or more mammals. The one or more mammals may be selected from the group comprising humans, apes, goats, sheeps, dogs, cows, mice, rats, cats, pigs, horses, or a combination thereof. The one or more samples may be from one or more humans. The one or more nucleic acid molecules may be detected without prior extraction and/or purification of the one or more nucleic acid molecules. The method may further comprise release/dissociation of the one or more nucleic acid molecules from one or more protective complexes. The one or more protective complexes may be selected from the group comprising cells, circulating cells, exosomes, lipid vehicles or protein complexes. The one or more solid supports may be selected from the group comprising beads, membranes, filters, slides, arrays, microarrays, chips, microtiter plates, and microcapillaries. The one or more TSPs further may comprise a linker. The linker may comprise an anchor group or hapten. The hapten may be biotin or digoxigenin. The bead may comprise a coated bead, magnetic bead, antibody-conjugated bead, or any combination thereof. The bead may be a streptavidin-coated magnetic bead. Detecting the one or more TSP-hybridized nucleic acid molecules or derivative thereof may comprise amplifying the one or more TSP-hybridized nucleic acid molecules or derivative thereof to produce one or more amplicons. The derivative of the one or more TSP-hybridized nucleic acid molecules may be selected from the group comprising a captured TSP-hybridized nucleic acid molecule, non-hybridized nucleic acid molecule, circularized nucleic acid molecule, nucleic acid copy molecules, amplicons or a combination thereof. The one or more nucleic acid molecules that may be hybridized to the one or more TSPs may comprise one or more RNA molecules. The one or more RNA molecules may comprise one or more microRNA (miRNA) molecules. The one or more RNA molecules may comprise one or more pre-miRNA, mature miRNA, or a combination thereof. The one or more nucleic acid molecules that may be hybridized to the one or more TSPs may comprise fragments of a DNA molecule, RNA molecule, or a combination thereof. The one or more TSPs hybridize to one or more nucleic acid molecules that may be 200 or fewer nucleotides or base pairs in length. The one or more TSPs hybridize to one or more nucleic acid molecules that may be 150 or fewer nucleotides or base pairs in length. The one or more TSPs hybridize to one or more nucleic acid molecules that may be 100 or fewer nucleotides or base pairs in length.

Disclosed herein is a system for analyzing one or more nucleic acid molecules comprising one or more TSPs, wherein the one or more TSPs may comprise a nucleic acid-specific portion; and instructions for hybridizing the one or more TSPs to one or more nucleic acid molecules, wherein (i) the one or more TSPs can hybridize to one or more nucleic acid molecules that may be less than or equal to 200 nucleotides or base pairs in length, and (ii) the length of the nucleic acid-specific portion may be less than or equal to the full length of the one or more nucleic acid molecules. The one or more molecules may be small RNA molecules. The one or more molecules may be miRNA. The system may further comprise one or more primers. The one or more primers may be less than or equal to 15 nucleotides in length. The system may further comprise a thermal cycler. The system may further comprise a sequencer. The system may further comprise a solid support. The solid support may comprise a bead. The bead may be a streptavidin-coated magnetic bead. The system may further comprise a separator. The separator may comprise a magnetic separator. The system may further comprise a computer. The system may further comprise a software. The one or more TSPs further may comprise a linker. The linker may comprise a label. The label may be biotin.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as Epicentre (Illumina), Bio-Rad, Stratagene (Agilent), New England Biolabs, Life Technologies, Sigma-Aldrich, Clontech Laboratories and others.

### REFERENCES

Asaga, S. et al. 2011. Clin. Chem. 57: 84-91.

Arroyo, J. D. et al. 2011. Proc. Natl. Acad. Sci. USA 108: 5003-8.

Beaucage, S. L. 2001. Curr. Med.Chem. 8: 1213-1244

Bryant, R. J. et al. 2012. Br. J. Cancer 106: 768-74.

Bushati, N., Cohen, S. M. 2007. Annu Rev. Cell Dev. Biol. 23: 175-205.

Chen, C. et al. 2005. Nucleic Acids Res. 33: e179.

Etheridge, A. et al. 2011. Mutat. Res. 717: 85-90.

Gallo, A. et al. 2012. PLoS One 7: e30679.

Gu, L-Q. et al. 2012. Expert Rev. Mol. Diagn. 12: 573-584.

Jost, R. et al. 2007. Biotechniques 43: 206-11.

Kim, D. J. et al. 2012a. J. Mol. Diagn. 14: 71-80.

Kim, Y. K. et al. 2012b. Mol. Cell. 46: 893-5.

Kroh, E. M. et al. 2010. Methods 50: 298-301.

Kumar, P. et al. 2011. RNA 17: 365-380.

McDonald, J. S. et al. 2011. Clin. Chem. 57: 833-40.

Mitsuhashi, M. et al. 2006. Clin. Chem. 52: 634-42.

Moltzahn, F. et al. 2011. Cancer Res. 71: 550-60.

Regan, P. M., Margolin, A. B. 1997. J. Virol. Methods. 64: 65-72.

Reid, G. et al. 2011. Crit. Rev. Oncol. Hematol. 80: 193-208.

Tanaka, A. et al. 2009. Anal. Sci. 25: 109-14.

Yolken, R. H. et al. 1991. Mol. Cell. Probes. 5: 151-6.

Zen, K., Zhang, C. Y. 2012. Med. Res. Rev. 32: 326-48.

Zhang, B., Farwell, M. A. 2008. J. Cell. Mol. Med. 12: 3-21.

Zheng, Z. et al. 2011. U.S. Pat. No., 7,927,798B2.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### Direct Quantification of miRNAs in Plasma without Capture Using TaqMan or miR-ID Detection

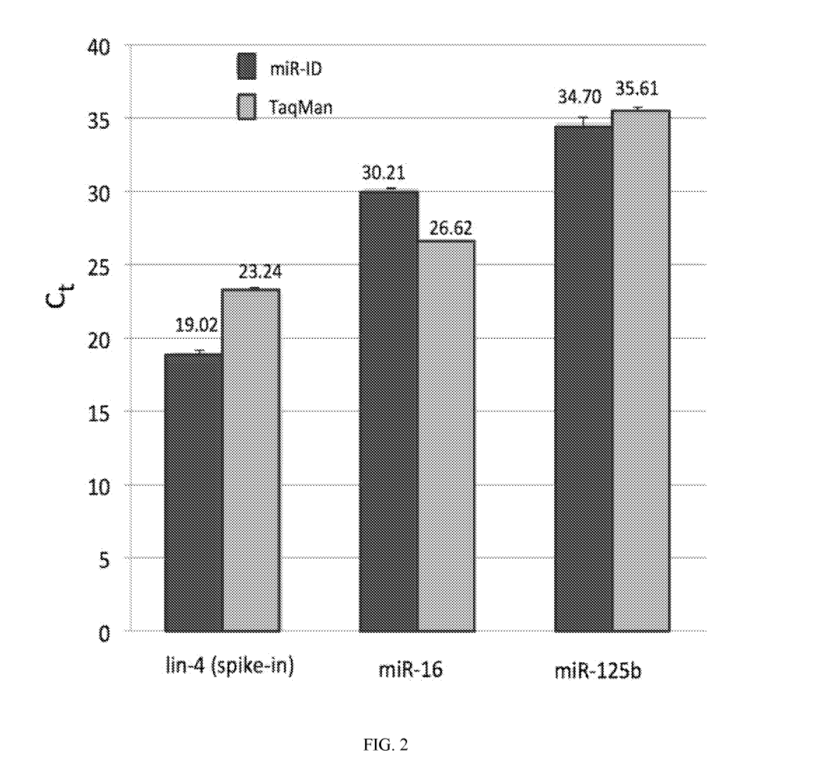

Frozen plasma specimens obtained from Biological Specialty Co. were thawed and filtered through a 1.2-mm filter to remove cells and cellular debris as recommended by Bryant et al. (2012). 24 μl of plasma was mixed with 1 μl of an RNAse inhibitor and incubated at 60° C. for 10 min. Next, an equal volume (25 μl) of a release/dissociation buffer and 1 fmol of synthetic lin-4 miRNA (as a spike-in control) was added and incubated at 25° C. for 1 hour. The samples were subsequently heated at 95° C. for 5 min and then centrifuged at 16,000 g for 4 min at 25° C. The supernatant was collected and 4 μl aliquots (each corresponding to 2 μl of the original plasma) were analyzed by miR-ID (Kumar et al. 2011) and TaqMan (Chen et al. 2005) RT-qPCR assays specific for hsa-miR-16-5p (miR-16), hsa-miR-125b-5p (miR-125b), cel-lin-4 (lin-4) and cel-miR-39 (cel-39) shown in Table 1. The results are shown in FIG. 2.

TABLE 1

| | | Sequence | SEQ |
|---|---|---|---|
| miRNA | miRBase ID | (5′-3′) | ID NO |
| cel-lin-4 | MIMAT0000002 | UCCCUGAGACCU | 1 |
| | | CAAGUGUGA | |
| cel-miR-39-3p | MIMAT0000010 | UCACCGGGUGUA | 2 |
| | | AAUCAGCUUG | |
| hsa-miR-16-5p | MIMAT0000069 | UAGCAGCACGUA | 3 |
| | | AAUAUUGGCG | |
| hsa-miR-125b-5p | MIMAT0000423 | UCCCUGAGACCC | 4 |
| | | UAACUUGUGA | |
| hsa-miR-148a-3p | MIMAT0000243 | UCAGUGCACUAC | 5 |
| | | AGAACUUUGU | |
| hsa-let-7d | MIMAT0000065 | AGAGGUAGUAGG | 6 |
| | | UUGCAUAGUU | |
| hsa-let-7g | MIMAT0000414 | UGAGGUAGUAGU | 7 |
| | | UUGUACAGUU | |
| hsa-miR-15b | MIMAT0000417 | UAGCAGCACAUC | 8 |
| | | AUGGUUUACA | |
| hsa-miR-106a | MIMAT0000103 | AAAAGUGCUUAC | 9 |
| | | AGUGCAGGUAG | |
| hsa-miR-142-3p | MIMAT0000434 | UGUAGUGUUUCC | 10 |
| | | UACUUUAUGGA | |
| hsa-miR-191 | MIMAT0000440 | CAACGGAAUCCC | 11 |
| | | AAAAGCAGCUG | |
| hsa-miR-301a | MIMAT0000688 | CAGUGCAAUAGU | 12 |
| | | AUUGUCAAAGC | |

For the TaqMan assays we used standard miRNA-specific kits (Applied Biosystems/Life Technologies) containing proprietary RT and PCR primers: TM 000391 (miR-16); TM 000449 (miR-125b); TM 000470 (miR-148a); TM 000258 (lin-4) and TM 000200 (cel-39). For the miR-ID assays we used RT and PCR primers shown in Table 2.

For the miR-ID assays, all RT reactions were done in multiplex, whereas for the TaqMan method they were performed in singleplex. qPCR reactions were performed in triplicate. Average Ct values are shown in FIG. 2.

TABLE 2

| | Primer Sequence | | SEQ |
|---|---|---|---|
| miRNA | (5′-3′) | Primer Type | ID NO |
| cel-lin-4 | GTCTCAGGGA | RT | 13 |
| | CTCAAGTGTGATCCCTGAG | PCR Forward | 14 |
| | AGGGATCACACTTGAGGTC | PCR Reverse | 15 |
| cel-miR-39-3p | ATTTACACCC | RT | 16 |
| | TAAATCAGCTTGTCACCGG | PCR Forward | 17 |
| | GTGACAAGCTGATTTACAC | PCR Reverse | 18 |
| hsa-miR-16-5p | CTGCTACGCC | RT | 19 |
| | AGCACGTAAATATTGGCG | PCR Forward | 20 |
| | CCAATATTTACGTGCTGC | PCR Reverse | 21 |
| hsa-miR-125b-5p | CAAGTTAGGG | RT | 22 |
| | GACCCTAACTTGTGATC | PCR Forward | 23 |
| | CACAAGTTAGGGTCTCA | PCR Reverse | 24 |
| hsa-miR-148a-3p | GCACTGAACA | RT | 25 |
| | TACAGAACTTTGTTCAGTGC | PCR Forward | 26 |
| | CTGAACAAAGTTCTGTAGTG | PCR Reverse | 27 |
| hsa-let-7d | CTACTACCTC | RT | 28 |
| | GTTAGAGGTAGTAGGTTGC | PCR Forward | 29 |
| | ACCTACTACCTCTAACTAT | PCR Reverse | 30 |
| hsa-let-7g | CTACTACCTC | RT | 31 |
| | GAGGTAGTAGTTTGTACAGTT | PCR Forward | 32 |
| | TGTACAAACTACTACCTCAAA | PCR Reverse | 33 |
| hsa-miR-15b | CCTGCACTGT | RT | 34 |
| | GCAGCACATCATGGTTTAC | PCR Forward | 35 |
| | AACCATGATGTGCTGCTATG | PCR Reverse | 36 |
| hsa-miR-106a | CCTGCACTGT | RT | 37 |
| | AGAAAAGTGCTTACAGTGC | PCR Forward | 38 |
| | CTGTAAGCACTTTTCTACC | PCR Reverse | 39 |
| hsa-miR-142-3p | CTACATCCAT | RT | 40 |
| | GTAGTGTTTCCTACTTTATG | PCR Forward | 41 |
| | AAAGTAGGAAACACTACATC | PCR Reverse | 42 |
| hsa-miR-191 | GGGATTCCGT | RT | 43 |
| | CAACGGAATCCCAAAAGC | PCR Forward | 44 |
| | TTTGGGATTCCGTTGCAG | PCR Reverse | 45 |
| hsa-miR-301a | CTGGCTTTGA | RT | 46 |
| | TGCAATAGTATTGTCAAAGC | PCR Forward | 47 |
| | TTGACAATACTATTGCACTG | PCR Reverse | 48 |

Example 1 demonstrated a method that released circulating miRNAs from plasma protective complexes and was compatible with conventional RT-qPCR detection methods. The method was performed without purifying the total RNA. The Ct values obtained by the TaqMan and miR-ID methods for the same levels of miRNAs were consistent with the relative sensitivities of these two methods as determined using standard dilution series of synthetic versions of the miRNAs. The lower-abundance miR-125b was close to the limit of detection by both methods with Ct˜35. However, this direct detection approach did not allow one to increase sensitivity by analyzing larger aliquots because the reaction volume for qPCR assays is typically limited to 15-20 μl.

### Example 2

### Quantification of miRNAs in Different Volumes of Plasma by miR-Direct using miR-ID Detection

Various volumes (25, 100 or 400 μl) of plasma sample #55, which was collected in an EDTA-containing tube from one individual, or 200 μL of plasma sample #M7259 collected either in heparin- or EDTA-containing tubes from a second individual (provided Biological Specialty Co.) were treated as follows to release the circulating miRNAs into solution. An equal volume of a lysis buffer was added to each plasma sample. Then a carrier RNA and 1 fmol cel-39 spike-in miRNA were added sequentially. The entire mixture was incubated at 25° C. for 1 hour. Following this incubation, 16 pmol of each of a set of target-specific oligonucleotide probes (TSPs) biotinylated at their 3′ ends (3-BioTEG, IDT), which were specific for miRNAs present in human blood (miR-16, miR-125b, miR-148a, let-7d, let-7g, miR-15b, miR106a, miRl42, miR-191 and miR-301a) or spiked-in miRNA cel-miR-39 (cel-39) (Table 3) were added. In addition to the sequences specific to the target miRNAs, these TSPs contain a universal 6-nt linker GTACTG at their 3′ ends. Hybridization of the TSPs to the RNA molecules comprised incubating the samples at 37° C. for 1 hour.

TABLE 3

| | | SEQ |
|---|---|---|
| miRNA | TSP Sequence (5′-3′) | ID NO |
| cel-lin-4 | CACTTGAGGTCTCAGGGTATCG | 49 |
| cel-miR-39-3p | GCTGATTTACACCCGGGTATCG | 50 |
| hsa-miR-16-5p | GCCAATATTTACGTGCTGCGTATCG | 51 |
| hsa-miR-125b-5p | CACAAGTTAGGGTCTCAGGTATCG | 52 |
| hsa-miR-148a-3p | GTTCTGTAGTGCACTGGTATCG | 53 |
| hsa-let-7d | TATGCAACCTACTACCTCTGTATCG | 54 |
| hsa-let-7g | CTGTACAAACTACTACCTCAGTATCG | 55 |
| hsa-miR-15b | TGTAAACCATGATGTGCTGCTGTATCG | 56 |
| hsa-miR-106a | CTGCACTGTAAGCACTTTTGTATCG | 57 |
| hsa-miR-142-3p | TCCATAAAGTAGGAAACACTACAGTATCG | 58 |
| hsa-miR-191 | CTGCTTTTGGGATTCCGTATCG | 59 |
| hsa-miR-301a | GCTTTGACAATACTATTGCACTGGTATCG | 60 |

For the capture step, 80 μg of streptavidin-coated magnetic beads (NEB) were added to the samples and were incubated at 37° C. for an additional 15 min. The beads were then separated on a magnetic rack (NEB) and washed twice with 500 μL of a washing buffer.

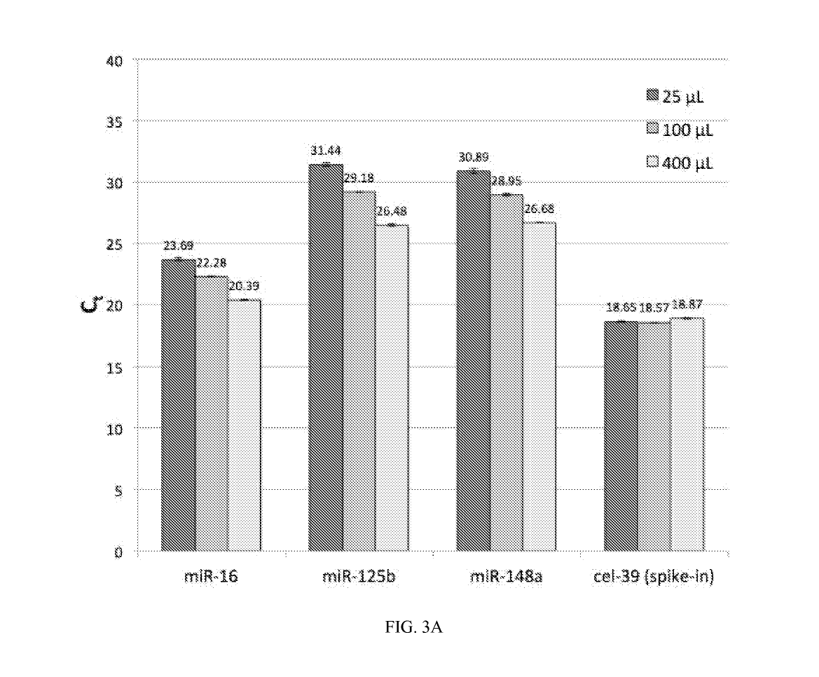

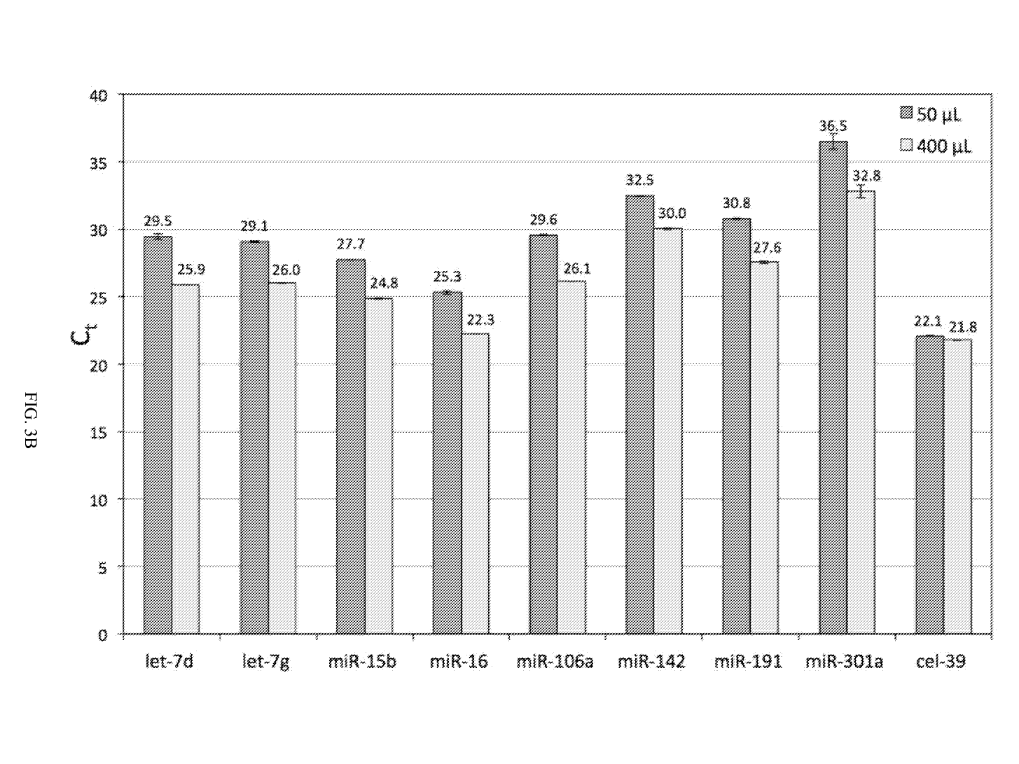

Release from beads and circularization of the captured miRNAs was performed by adding 10 μL of a release buffer solution with 5 μL of CircLigase II (Epicentre) and incubating the samples at 60° C. for 15 min. Following the ligation reaction, the beads were separated on the magnetic rack and the entire solution phase (about 8 μl) was recovered and used in the miR-ID RT-qPCR assay. The miR-ID RT-qPCR assays were performed as described in Kumar et al. (2011) using miRNA-specific primers shown in Table 2. In these assays, qPCR reactions were performed in triplicate and the resulting average Cₜ values are shown in FIG. 3A-3B. In FIG. 3A, the dark grey bars represent 25 μL assay volumes; medium grey bars represent 100 μL assay volumes; and light grey bars represent 400 μL assay volumes. In FIG. 3B, the dark grey bars represent 50 μL assay volumes and light grey bars represent 400 μL assay volumes.

In this example, we demonstrated the detection of circulating and spike-in miRNAs by the miR-Direct method using miR-ID detection. We found that the miRNA levels measured increased in proportion to the starting volume of plasma, with about a 2-Cₜ decrease for every 4-fold increase of plasma volume while the Cₜ for the cel-39 spike-in control remained about the same (FIG. 3A-B). The miR-Direct method enabled detection of both circulating and spiked-in miRNAs and was scalable over a wide-range of starting plasma volumes.

### Example 3

### Quantification of miRNAs in Various Plasma Samples by miR-Direct using miR-ID Detection

400 μl of each of plasma sample from 11 healthy donors were analyzed using the miR-Direct capture with miR-ID detection specific for the circulating miRNAs hsa-miR-16, hsa-miR-125b and hsa-miR-148a as well as a spike-in control, cel-miR-39 as described in Example 2.

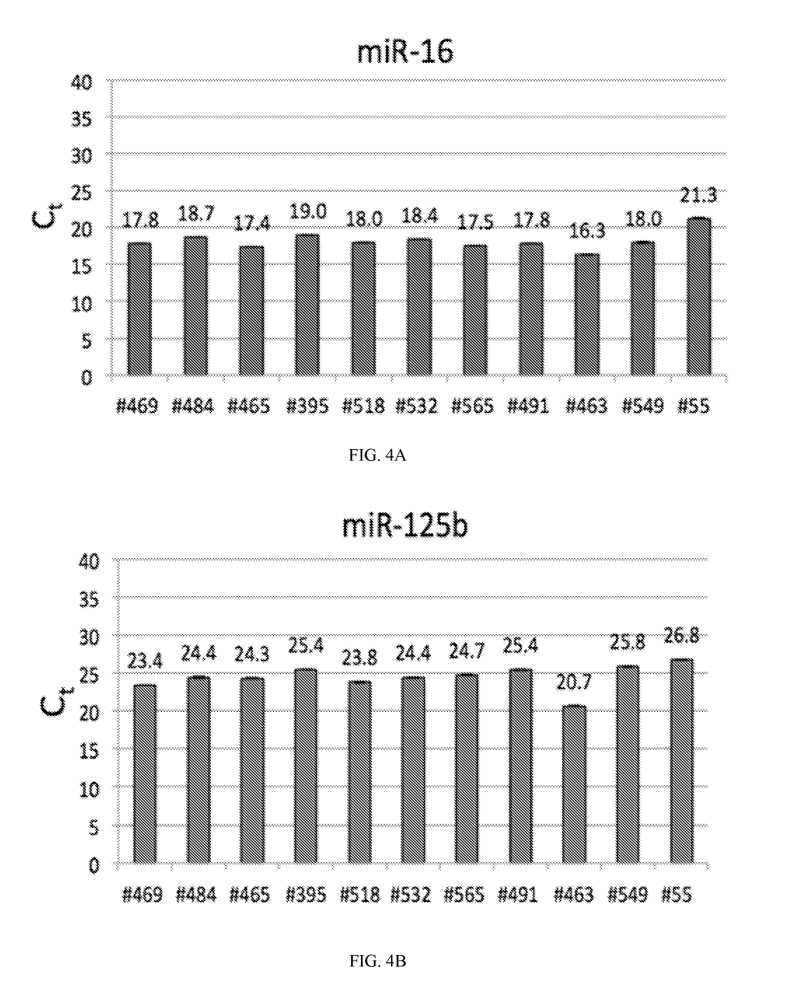

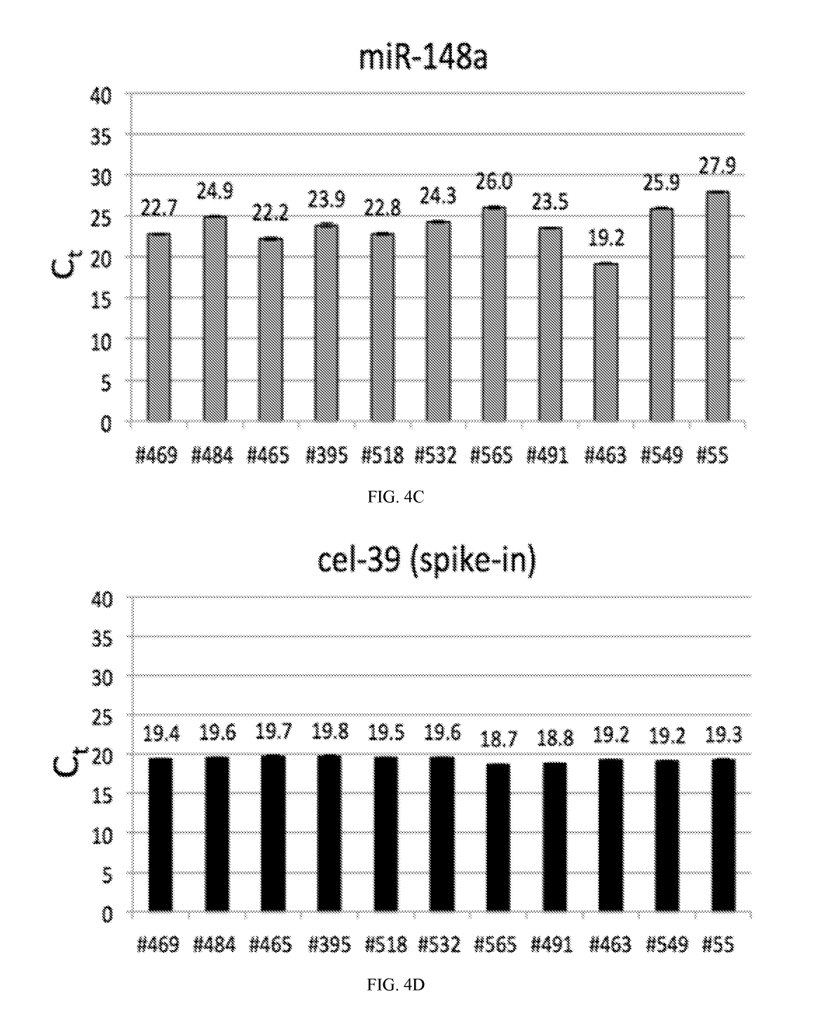

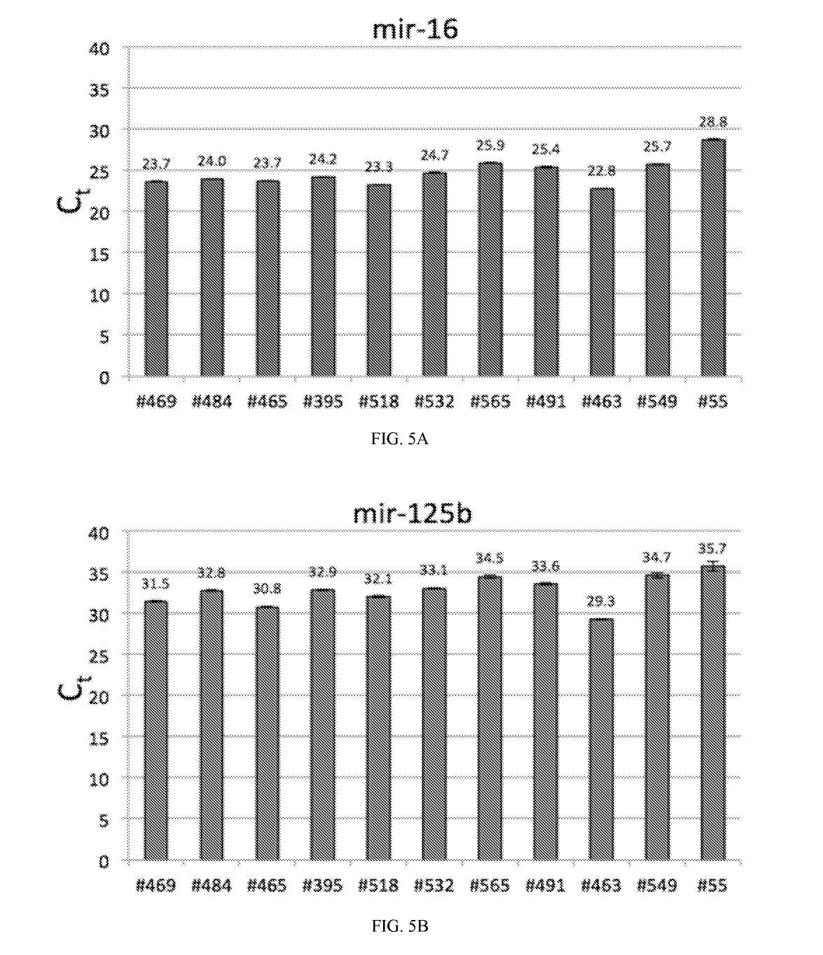

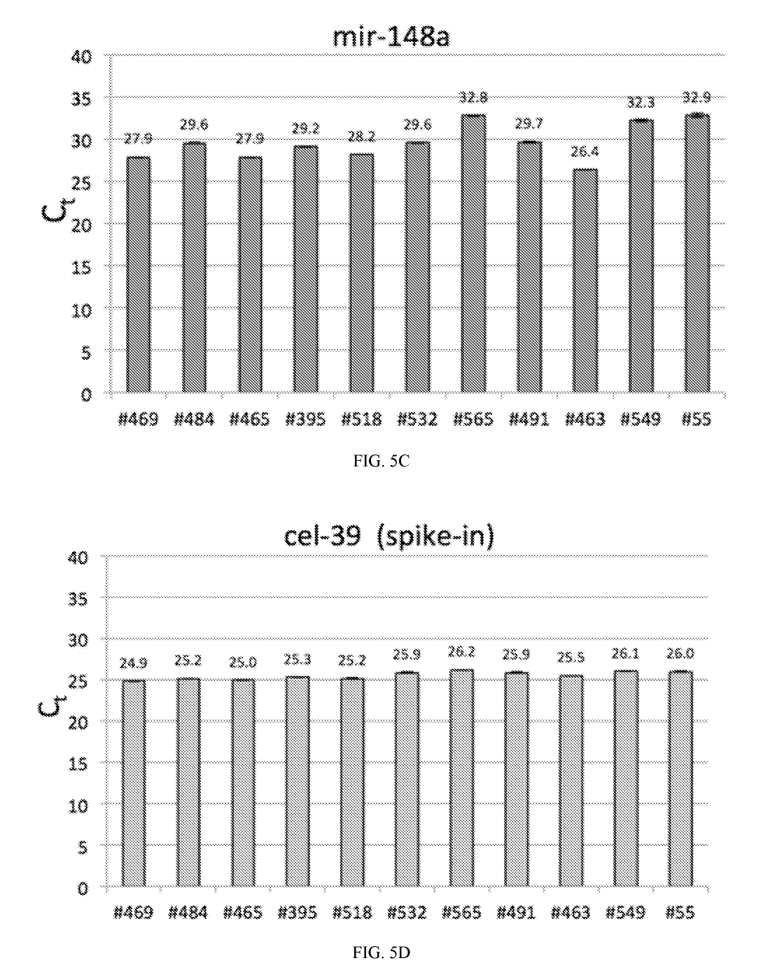

In this example, we observed that the levels of the circulating miRNAs varied by 1-3 Cₜ units among the various plasma samples while the spike-in control remained constant (FIG. 4). To verify the Cₜ variations measured by miR-Direct, we compared the results obtained by miR-Direct and conventional miR-ID, which uses column-purified total RNA (see Example 4 and FIG. 5).

### Example 4

### Quantification of miRNAs in various Plasma Samples using Total RNA Isolated from Plasma by Column Purification, with miR-ID Detection

Total RNA was extracted from 100 μl of each of the plasma specimens used in Example 3 using the miRNeasy kit (Qiagen) according to the protocol described by Kroh et al. (2010), with the spike-in control miRNAs added to the master mix containing QIAzol and MS2 carrier RNA (Roche). Using inputs of total RNA equivalent to 8 μl of plasma for each RT-qPCR assay, we performed the standard miR-ID assays (Kumar et al. 2011) for the chosen circulating miRNAs (hsa-miR-16, hsa-miR-125b and hsa-miR-148a) and the spike-in control cel-miR-39 as described in Example 1. The RT step was run in multiplex reaction (e.g., all miRNAs together, but with specimens remaining separate) and the qPCR reactions were performed in triplicate. The resulting average Cₜ values are shown in FIG. 5. The conventional miR-ID assays using total RNA from 8-μl plasma were found to be about 50-fold (-5.5 Ct) less sensitive than the miR-IDirect assay from Example 3, where the volume of plasma input was 50-fold higher (400 μl) (compare FIGS. 4 and 5).

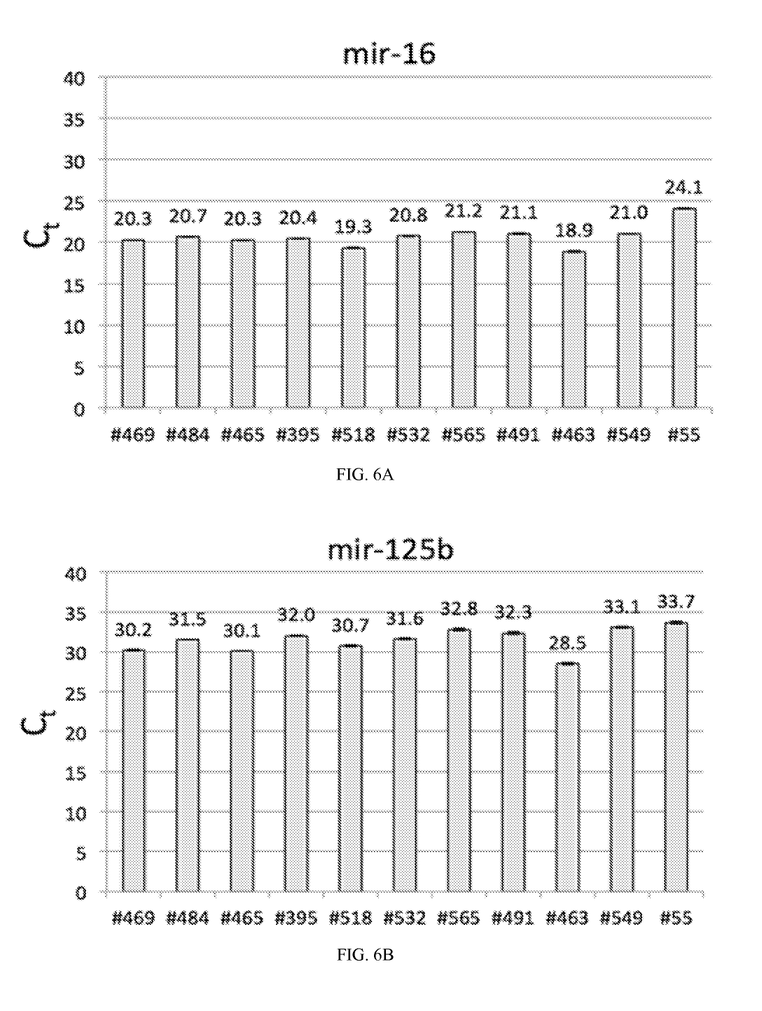

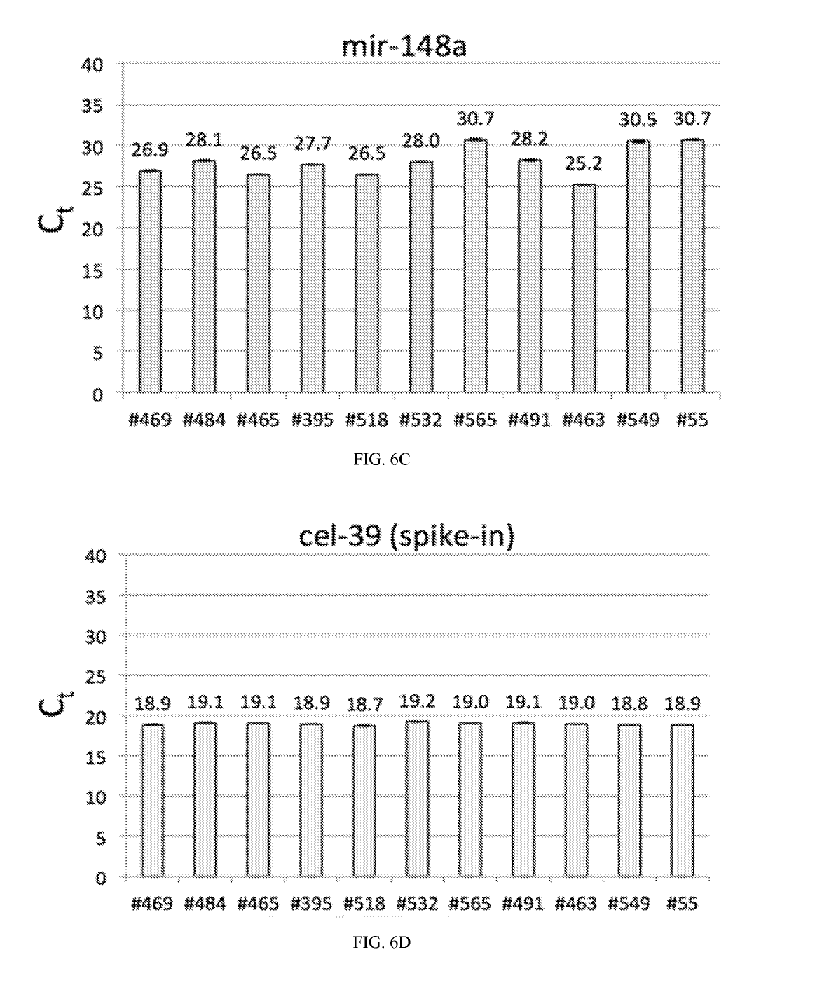

In this example, we found that the miRNA expression profiles of the various specimens were consistent for both miR-ID methods, with or without prior isolation of total RNA (compare FIGS. 4 and 5). The patterns of variability of miRNA expression profiles among the various specimens were confirmed by TaqMan assays performed using the same total plasma RNA samples (see Example 5 and FIG. 6).

### Example 5

### Quantification of miRNAs in Various Plasma Samples using total RNA Isolated from Plasma by Column Purification, with TaqMan Detection

Singleplex TaqMan assays (Chen et al. 2005) specific for the same circulating and spike-in miRNAs as in Example 4 were run using the same column-purified total plasma RNA in amounts corresponding to 4 μl of plasma for each assay. qPCR reactions were performed in triplicate. The resulting average Cₜ values are shown in FIG. 6.

The Cₜ values obtained by miR-ID (FIG. 5) and TaqMan (FIG. 6) detection for the same samples and the same miRNAs were found to be consistent with the relative sensitivities of these two methods previously determined using standard dilution series of these miRNAs.

### Example 6

### Quantification of miRNAs in Plasma by miR-Direct using Various miRNA Elution Conditions, with TaqMan Detection

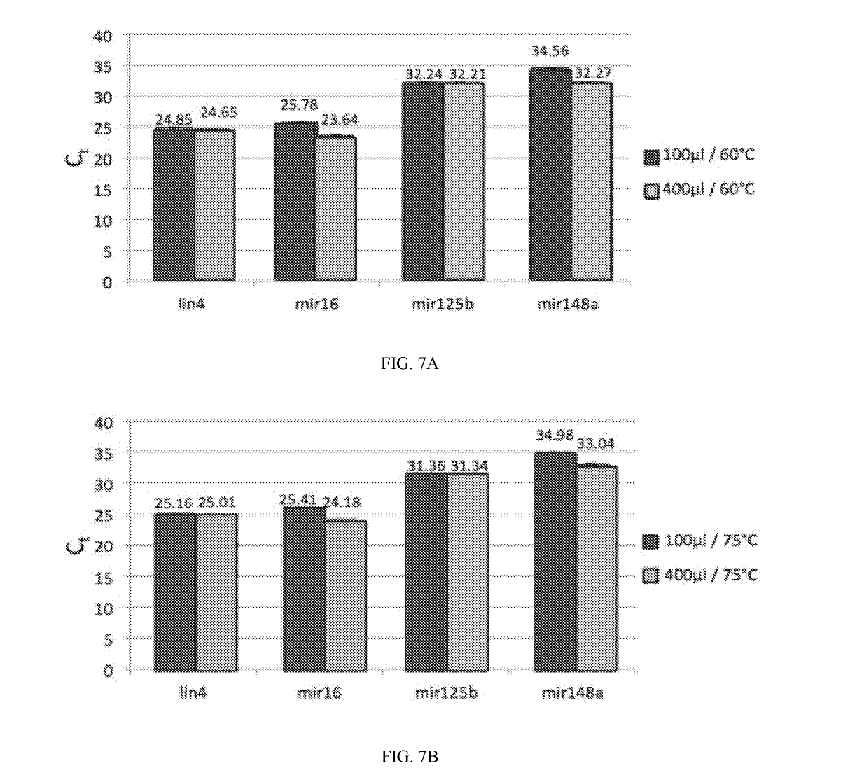

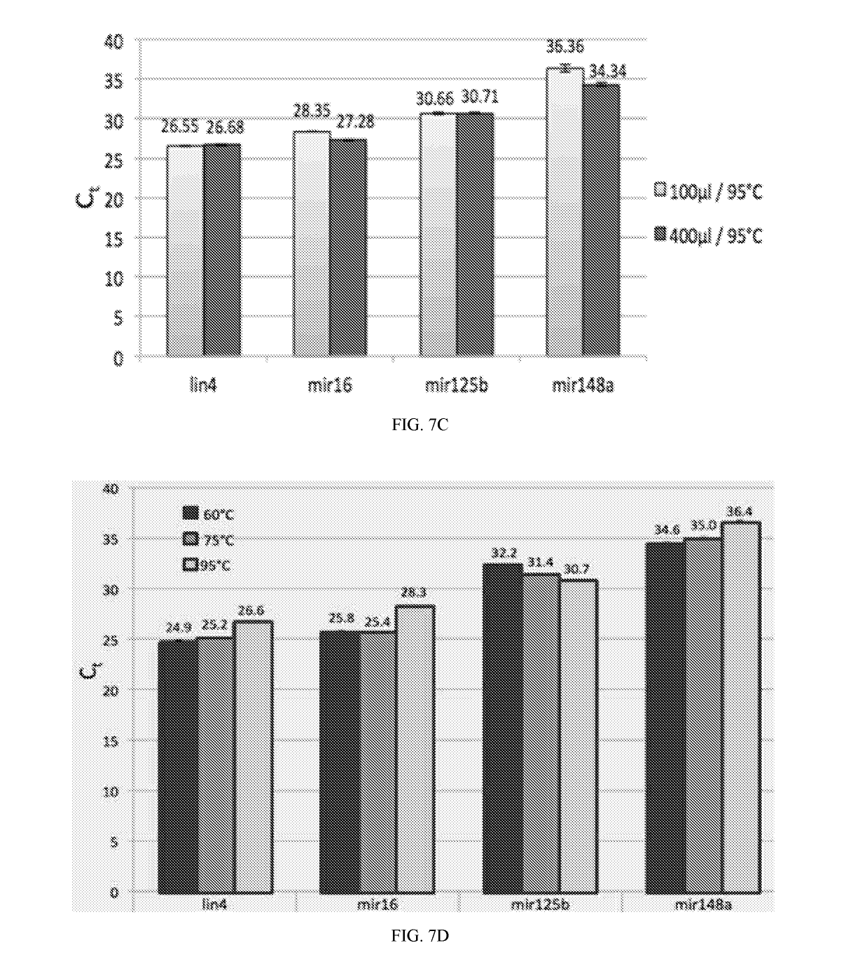

100 and 400 μL aliquots of plasma (sample #55) were treated by the release and capture procedure described in Example 2 except that cel-lin4 miRNA was used as a spike-in control in place of cel-39 miRNA and the dissociation procedure for release of the captured miRNAs into solution differed from the one used for miR-ID assay as follows. Following the capture of the miRNAs on the magnetic beads and washing, the RNAs were eluted by incubation at either 60° C. for 15 min, 75° C. for 5 min, or 95° C. for 5 min in 12 μL of deionized water. The eluted miRNAs were then assayed by singleplex TaqMan RT-qPCR according to the standard protocol (Chen et al. 2005). qPCR reactions were performed in triplicate. The resulting average Cₜ values are shown in FIG. 7.

In this example, we demonstrated the detection of circulating and spike-in miRNAs by the miR-Direct method using TaqMan detection. As with Example 2, where detection of the captured miRNAs was by miR-ID, we found that the measured circulating miRNA levels increased in proportion to the starting volume of plasma, with about a 2-Cₜ decrease for every 4-fold increase of plasma volume for miR-16 and mir-148a, while the Cₜ for the lin4 spike-in control remained about the same as expected (see FIG. 7A-C). However, for miR-125b, we did not observe a decrease in Cₜ for the higher plasma volume. We speculated that the elution of the linear miRNAs (in contrast to circular ones) may be reversible and, therefore, incomplete and inconsistent. To determine whether a higher elution temperature would result in the better recovery of the captured miRNAs, we compared the amount of the detected miRNAs eluted at 60, 75 and 95° C. (FIG. 7D). We found that increasing the elution temperature did not result in consistent improvement of the miRNA detection (except for miR-148a).

### Example 7

### Quantification of miRNAs in Various Plasma Samples by miR-Direct: Comparison of miR-ID vs. TaqMan Detection

100 μL aliquots of plasma (sample #55) were processed according to Steps 1 to 3 of the miR-Direct protocol (see FIG. 1) as described in Example 2 (for miR-ID detection) and Example 6 (for TaqMan detection). The miRNAs (miR-16, miR-125b and miR-148a) captured on magnetic beads miRNAs were eluted at 60° C. and then either circularized and assayed by miR-ID (as described in Example 2), or kept in solution in linear form and assayed by the TaqMan procedure (as described in Example 6). In contrast to miR-ID, for which all RT reactions were done in multiplex, the RT reactions for the TaqMan method were performed in singleplex because of the incompatibility of the supplied RT primers for these miRNAs. qPCR reactions were performed in triplicate. The resulting average Cₜ values are shown in Table 4.

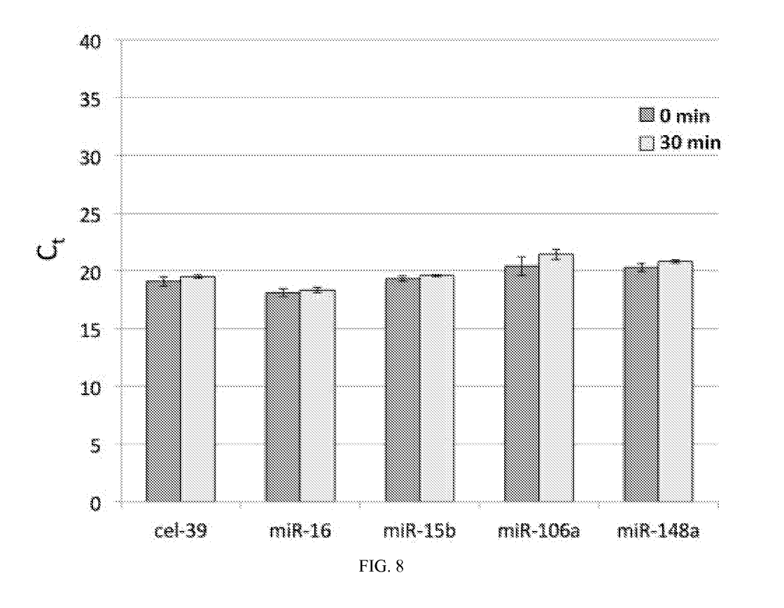

We found that for the miR-Direct approach, TaqMan detection was less sensitive than miR-ID detection for circulating miR-16, miR-125b, and miR-148a (by 3.5, 3.0 and 5.6 Ct, respectively, for the same starting amount of plasma) (see Table 4). This advantage of miR-ID detection is specific to the miR-Direct approach; the sensitivity of conventional TaqMan detection for miR-16 and miR-148a was lower by 1-2 Cₜ according to standard dilution curves, while for miR-125b the sensitivity was the same for both methods. This advantage of miR-ID over TaqMan for the miR-Direct approach may be related to the procedure for eluting miRNAs captured on the immobilized probes. For the miR-ID assay, the elution of miRNAs occurs simultaneously with their circularization. Steric constraints in circular miRNAs of 21-22 nt presumably restrict their ability to form duplexes longer than 10 nt (Kumar et al. 2011), thus reducing their affinity to the hybridization probes and inhibiting their re-hybridization with miRNAs as demonstrated in FIG. 8. FIG. 8 shows the results of an experiment in which 200 μl of a single plasma sample was analyzed using miR-Direct capture with miR-ID detection specific for the circulating miRNAs hsa-miR-16, hsa-miR-15b, has-miR-106a and hsa-miR-148a as well as a spike-in control, cel-miR-39 as described in Example 2. The quantity of the miRNAs was determined after 0 minutes or 30 minutes recovery of the captured miRNAs from the beads after miRNA dissociation and circularization. In this experiment, we observed no appreciable difference in detection of circularized miRNAs when they were separated from beads immediately after circularization (0 minutes) versus after 30 minutes incubation at ambient temperature (see FIG. 8). As shown in FIG. 8, dark grey bars represent 0 minutes of recovery and light grey bars represent 30 minutes of recovery.

In contrast, the elution of the linear miRNAs used in TaqMan detection may be reversible and, therefore, incomplete and inconsistent. In addition, the higher-temperature elution conditions used in the TaqMan procedure in Example 6 may also induce partial degradation of miRNAs (which could make them undetectable) or release some of the immobilized probes into solution (which could interfere with the RT-qPCR detection).

TABLE 4

| | Average Cₜ | | |
|---|---|---|---|
| miRNAs | miR-ID | TaqMan | ΔCₜ |
| miR-16 | 22.3 | 25.8 | −3.5 |
| miR-125b | 29.2 | 32.2 | −3 |
| miR-148a | 29.0 | 34.6 | −5.6 |

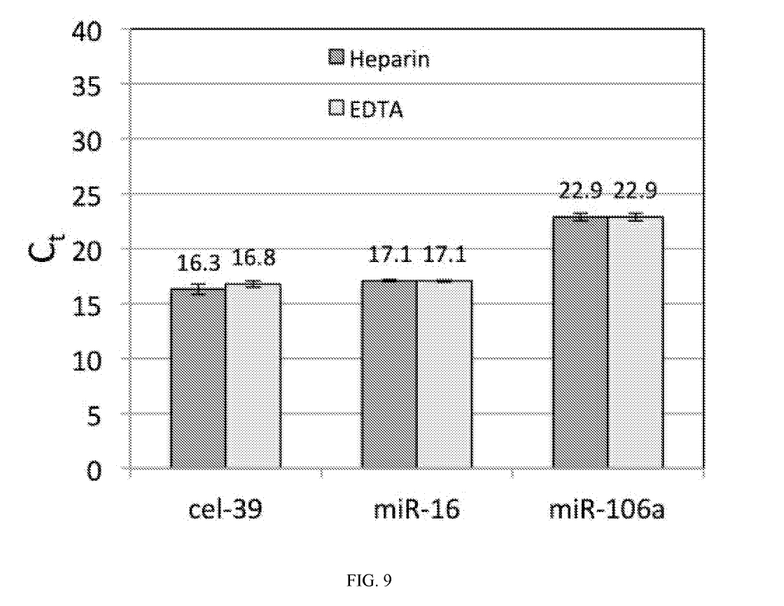

In another experiment, 200 μl aliquots of plasma samples that were collected into either heparin or EDTA tubes from a single individual were analyzed using miR-Direct capture with miR-ID detection specific for the circulating miRNAs hsa-miR-16 and hsa-miR-106a as well as for a spike-in control, cel-miR-39, as described in Example 2. We found no appreciable difference in levels of miRNA detected in the EDTA-treated plasma samples (see FIG. 9, light grey bars) in comparison to the heparin-treated samples (see FIG. 9, dark grey bars) despite the fact that heparin is known to be a strong inhibitor of conventional RT-PCR assays using total RNA isolated from plasma or serum (such as the TaqMan RT-qPCR microRNA assay).

What is claimed is:

1. A method for analyzing one or more nucleic acid molecules, comprising:
a. contacting in solution one or more samples comprising one or more nucleic acid molecules with one or more target-specific oligonucleotide probes (TSPs) to produce one or more TSP-hybridized nucleic acid molecules, wherein:
i. the one or more TSPs hybridize to one or more nucleic acid molecules that are 200 or fewer nucleotides or base pairs in length;
ii. the one or more TSPs comprise a nucleic acid-specific portion that hybridizes to at least a portion of the one or more nucleic acid molecules;
b. capturing the TSP-hybridized nucleic acids on a solid support to produce captured nucleic acid molecules;
c. removing one or more analytes and other solution components from the sample, wherein the one or more analytes are not hybridized to the one or more TSPs;
d. releasing the captured nucleic acid molecules into solution to produce released nucleic acid molecules, wherein the nucleic acid molecules are circularized following release; and
e. detecting the circularized nucleic acid molecules.

2. The method of claim 1, wherein the TSP does not serve as a template for 3′-end extension of the nucleic acid molecule.

3. The method of claim 1, wherein producing the released nucleic acid molecules comprises use of one or more ligases.

4. The method of claim 3, wherein the ligase is a thermostable ligase.

5. The method of claim 4, wherein the thermostable ligase is a CircLigase or CircLigase II.

6. The method of claim 1, wherein producing the released nucleic acid molecules comprises heating the sample to a temperature greater than a melting temperature (Tm) of the one or more TSPs.

7. The method of claim 6, wherein the temperature is greater than or equal to 50° C., 55° _{C.,} 60° _{C.,} 65° C., 67° _{C.,} 70° C., 72° C., 75° C., 77° C., or 80° C.

8. The method of claim 1, wherein detecting the one or more released nucleic acid molecules comprises reverse transcribing the one or more released nucleic acid molecules or derivative thereof to produce one or more nucleic acid copy molecules that are complements of the one or more TSP-hybridized nucleic acid molecules or a derivative thereof.

9. The method of claim 1, wherein detecting the one or more TSP-hybridized nucleic acid molecules comprise use of one or more 5′-overlapping PCR primer pairs.

10. The method of claim 9, wherein the length of the one or more primers is less than or equal to about 12, 11, 10, 9, 8, 7, or 6 nucleotides.

11. The method of claim 1, wherein the one or more solid supports are selected from the group comprising beads, membranes, filters, slides, arrays, microarrays, chips, microtiter plates, and microcapillaries.

12. The method of claim 11, wherein the bead comprises a coated bead, magnetic bead, antibody-conjugated bead, or any combination thereof.

13. The method of claim 11, wherein the bead is a streptavidin-coated magnetic bead.

14. The method of claim 1, wherein the one or more nucleic acid molecules of the TSP-hybridized nucleic acid molecules comprise one or more RNA molecules.

15. The method of claim 14, wherein the one or more RNA molecules comprise one or more microRNAs (miRNA), pre-miRNAs, or a combination thereof.

16. The method of claim 1, wherein the one or more TSPs hybridize to one or more nucleic acid molecules that are less than about 150, less than about 100, less than about 70, less than about 50, or less than about 40 nucleotides or base pairs in length.

17. A kit for identifying, detecting, or quantifying one or more nucleic acids, the kit comprising:
a. one or more target-specific probes (TSPs) comprising a nucleic acid-specific portion that selectively hybridizes to one or more nucleic acids, wherein the one or more TSPs does not serve as a template for 3′-end extension of the one or more nucleic acids;
b. one or more primers; and
c. a CircLigase
d. optionally, one or more buffers or solutions.

18.-23. (canceled)

24. The kit of claim 22, wherein the RT primer has a length of less than or equal to about 12, about 10, about 9, about 8, about 7, or about 6 nucleotides.

25. (canceled)

26. The kit of claim 17, further comprising one or more solid supports selected from a group consisting of beads, membranes, filters, slides, arrays, microarrays, chips, microtiter plates, and microcapillaries.

27. (canceled)

28. (canceled)

29. The kit of claim 28, wherein the RNA is a microRNA (miRNA), pre-miRNA, or a combination thereof.
